(19) United States
(12) Patent Application Publication
(10) Pub. No.: US 2023/0312573 A1
(43) Pub. Date: Oct. 5, 2023

(54) NOVEL SALTS, CRYSTALS, AND CO-CRYSTALS

(71) Applicant: INTRA-CELLULAR THERAPIES, INC., New York, NY (US)

(72) Inventor: Peng LI, New Milford, NJ (US)

(73) Assignee: INTRA-CELLULAR THERAPIES, INC., New York, NY (US)

(21) Appl. No.: 18/043,959

(22) PCT Filed: Sep. 3, 2021
(86) PCT No.: PCT/US2021/071366

§ 371 Date: Mar. 3, 2023

(51) Int. Cl. C07D 471/16

(52) U.S. Cl. CPC C07D 471/16; C07B 2200/13

(57) ABSTRACT

The disclosure provides salts and crystal forms of a substituted heterocycle fused gamma-carboline, the manufacture thereof, pharmaceutical compositions thereof, and use thereof, e.g., in the treatment of diseases or abnormal conditions involving or mediated by the 5-HT_{2A} receptor, serotonin transporter (SERT), and/or dopamine D₁/D₂ receptor signaling pathways.

### CROSS-REFERENCE TO RELATED APPLICATIONS

This international patent application claims priority to, and the benefit of, U.S. Provisional Application Ser. No. 63/075,019, filed on Sep. 4, 2020, the contents of which are hereby incorporated by reference in its entirety.

### FIELD

This disclosure relates to certain novel salts and crystal forms of a substituted heterocycle fused gamma-carboline, the manufacture thereof, pharmaceutical compositions thereof, and use thereof, e.g., in the treatment of diseases or abnormal conditions involving or mediated by the 5-HT_{2A} receptor, serotonin transporter (SERT), and/or dopamine D₁/D₂ receptor signaling pathways.

### BACKGROUND

Psychosis, particularly schizophrenia and schizoaffective disorder, affects an estimated 1-2% of the population worldwide. Schizophrenia is comprised of three phases: prodromal phase, active phase and residual phase. Prodromal phase is an early phase wherein subclinical signs and symptoms are observed. These symptoms may include loss of interest in usual pursuits, withdrawal from friends and family members, confusion, trouble with concentration, feeling of listlessness and apathy. Active phase is characterized by exacerbations of positive symptoms such as delusions, hallucinations and suspiciousness. Residual phase is characterized by negative symptoms such as emotional withdrawal, passive social withdrawal, and stereotyped thinking; and general psychopathological symptoms including active social avoidance, anxiety, tension, and somatic concerns. Residual phase symptoms are also often accompanied by depression, cognitive dysfunction and insomnia. Collectively, these residual phase symptoms (and especially the negative symptoms) are not well-treated by many antipsychotic drugs currently available on the market and therefore are usually observed after the active phase symptoms have subsided after treatment. This phase of the illness is when patients would like to return to more productive and fulfilling lives, but since the residual negative symptoms and cognitive impairment are not properly treated, it frustrates the return to such a function. There remains an urgent need for anti-psychotic agents, which can treat not just the active or acute phase symptoms, but also the residual phase symptoms of psychosis, e.g., schizophrenia. In addition, there is a need for medications to treat these symptoms that are free from undesirable side effects caused by off-target interactions with histamine H1 and muscarinic acetylcholine receptor systems.

Other central nervous system diseases and disorders which remain very difficult to effectively treat include dementia, such as Alzheimer's disease, and symptoms associated with or caused by the underlying dementia, such as anxiety, agitation, aggression, cognitive dysfunction, and memory loss. Even drugs that are effective in treating behavioral problems, including anxiety, agitation, and aggression, in other patients have not been effective in treating such problems in dementia patients, likely due to differences in the underlying etiology.

Substituted heterocycle fused gamma-carbolines are known to be agonists or antagonists of 5-HT₂ receptors, particularly 5-HT_{2A} receptors, in treating central nervous system disorders. These compounds have been disclosed in U.S. Pat. Nos. 6,548,493; 7,238,690; 6,552,017; 6,713,471; 7,183,282; U.S. RE39680, and U.S. RE39679, as novel compounds useful for the treatment of disorders associated with 5-HT_{2A} receptor modulation such as obesity, anxiety, depression, psychosis, schizophrenia, sleep disorders, sexual disorders migraine, conditions associated with cephalic pain, social phobias, gastrointestinal disorders such as dysfunction of the gastrointestinal tract motility, and obesity.

WO 2008/112280 and US2010/0113781, incorporated by reference herein in their entireties, disclose methods of making substituted heterocycle fused gamma-carbolines and uses of these gamma-carbolines as serotonin agonists and antagonists useful for the control and prevention of central nervous system disorders such as addictive behavior and sleep disorders.

WO/2009/145900 and US2011/0071080, incorporated by reference herein in their entireties, disclose use of particular substituted heterocycle fused gamma-carbolines for the treatment of a combination of psychosis and depressive disorders as well as sleep, depressive and/or mood disorders in patients with psychosis or Parkinson's disease. In addition to disorders associated with psychosis and/or depression, this reference discloses the use of these compounds at a low dose to selectively antagonize 5-HT_{2A} receptors without affecting or minimally affecting dopamine D₂ receptors, thereby useful for the treatment of sleep disorders without the side effects of the dopamine D₂ pathways or side effects of other pathways (e.g., GABAA receptors) associated with conventional sedative-hypnotic agents (e.g., benzodiazepines) including but not limited to the development of drug dependency, muscle hypotonia, weakness, headache, blurred vision, vertigo, nausea, vomiting, epigastric distress, diarrhea, joint pains, and chest pains.

WO 2015/085004 and US 2016/0310502, incorporated by reference herein in their entireties, also disclose that these compounds are particularly and unexpectedly effective in treating the residual symptoms, such as negative symptoms, of schizophrenia.

Additional therapeutic uses, including post-traumatic stress disorder, impulse control disorder, intermittent explosive disorder, dementia and disorders associated with dementias, acute depression and acute anxiety, have been disclosed for these compounds as well. See WO 2013/155504, US 2015/0072964, WO 2013/155506, US 2015/0080404, WO 2019/178484, and US 2021/00600009, each of which is incorporated by reference herein in their entireties.

WO 2009/114181 and US2011/112105, incorporated by reference herein in their entireties, disclose methods of preparing toluenesulfonic acid addition salt crystals of particular substituted heterocycle fused gamma-carbolines, e.g., toluenesulfonic acid addition salt of 4-((6bR,10aS)-3-methyl-2,3,6b,9,10,10a-hexahydro-1H-pyrido[3′,4′: 4,5]pyrrolo[1,2,3-de]quinoxalin-8(7H)-yl)-1-(4-fluorophenyl)-1-butanone. Additional salt forms and co-crystal forms are disclosed in WO 2017/172784, US 2019/0112309, WO 2017/172811, US 2019/0112310, WO 2019/067591, US 2020/247805, WO 2019/236889, and US 2020/1057100, each of which are incorporated by reference herein in their entireties.

WO 2011/133224 and US2013/202692 disclose prodrugs/metabolites of substituted heterocycle fused gamma-carboline for improved formulation, e.g., extended/controlled release formulation. This application discloses that heterocycle fused gamma-carboline N-substituted with a 4-fluorophenyl-(4-hydroxy)-butyl moiety are shown to have high selectivity for the serotonin transporter (SERT) relative to the heterocycle fused gamma-carboline containing 4-fluorophenylbutanone. The hydroxy group on these compounds, however, is inter-converted to and from the ketone within the plasma and the brain, allowing it to serve as a reservoir for the 4-fluorophenylbutanone drug. While substituted heterocycle fused gamma-carbolines and their uses are known, our inventors have surprisingly found that particular substituted heterocycle fused gamma-carbolines, while less active in in-vitro tests, are inter-converted between these less active compounds and the highly active ketone drug within the plasma and the brain. Our inventors have further provided prodrugs of particular substituted heterocycle fused gamma-carbolines that have altered pharmacokinetic profile, e.g., altered mechanisms and/or rate of absorption and distribution, and therefore may be useful for an improved formulation and/or for controlling the duration of the effect of the drug in the body (e.g., for sustained- or controlled release).

WO 2013/155505 and US 2015/0079172 disclose compounds which block the in vivo inter-conversion between the hydroxy and the ketone, by incorporating an alkyl substituent on the carbon bearing the hydroxyl group, thus yielding compounds which antagonize 5-HT_{2A} receptors and also inhibit serotonin re-uptake transporter.

A particularly preferred compound disclosed in the aforementioned references is 1-(4-fluoro-phenyl)-4-((6bR,10aS)-3-methyl-2,3,6b,9,10,10a-hexahydro-1H,7H-pyrido[3′,4′:4,5]pyrrolo[1,2,3-de]quinoxalin-8-yl)-butan-1-one (sometimes referred to as 4-((6bR,10aS)-3-methyl-2,3,6b,9,10,10a-hexahydro-1H-pyrido[3′,4′:4,5]pyrrolo[1,2,3-de]quinoxalin-8(7H)-yl)-1-(4-fluorophenyl)-1-butanone. This compound is also known as ITI-007, and as lumateperone. Lumateperone tosylate is currently approved in the United States by the Food & Drug Administration for the treatment of schizophrenia under the brand name CAPLYTA®. It is also undergoing or has undergone human clinical studies evaluating its effectiveness in the treatment of bipolar depression and dementia. Lumateperone has the following structure:

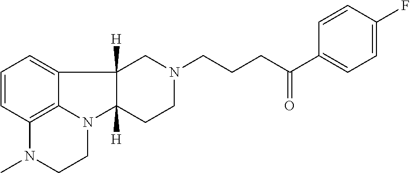 [SMILES: CN1CCN2c3c(cccc31)[C@@H]1CN(CCCC(=O)c3ccc(F)cc3)CC[C@@H]12]

Lumateperone is a potent 5-HT_{2A} receptor ligand (Ki=0.5 nM) with strong affinity for dopamine (DA) D2 receptors (Ki=32 nM) and the serotonin transporter (SERT) (Ki=62 nM) but negligible binding to receptors (e.g., H1 histaminergic, 5-HT2C, and muscarinic) associated with cognitive and metabolic side effects of antipsychotic drugs.

WO2015/154025 and US2017/0183350, incorporated by reference herein in their entireties, disclose the major routes of metabolism of lumateperone as N-demethylation catalyzed by CYP 3A4, and ketone reduction catalyzed by ketone reductase. N-dealkylation by cytochrome oxidase enzymes is known to occur via an initial oxidation of one or more of the carbon atoms alpha to the nitrogen atom. The family of enzymes that catalyze ketone reduction is large and varied, and the mechanism has not been absolutely elucidated. These references further disclose generic deuterated heterocycle fused gamma carbolines for the purpose of reducing metabolic degradation by partially limiting metabolism of the ketone and/or the N-methyl substituent.

WO 2017/165843 and US 2019/0231780, incorporated by reference herein in their entireties, further disclose three particular deuterated derivatives of lumateperone that were found to be particularly potent and having significantly reduced metabolic degradation. WO 2019/183546, and US 2021/0008065, each incorporated by reference herein in their entireties, further disclose additional deuterated derivatives of lumateperone. These deuterated compounds are disclosed as generally being amenable to synthesis in both free base form and in toluenesulfonic acid addition salt form. The following specific compounds are exemplified in these references:

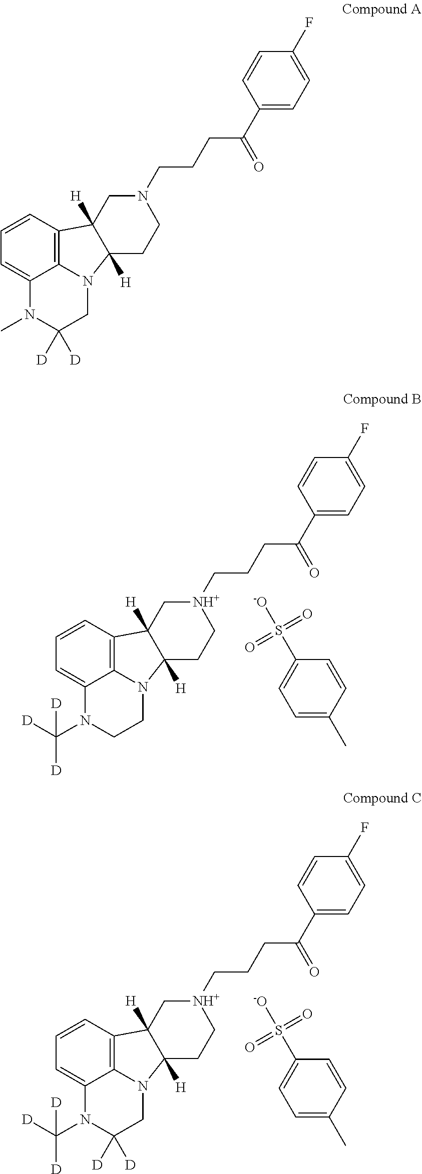

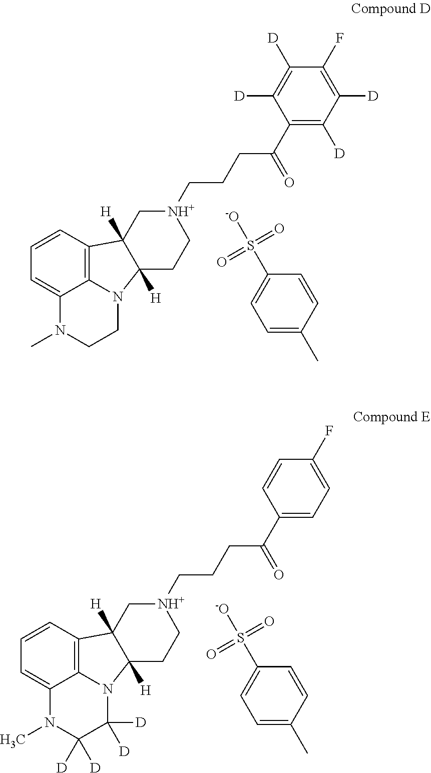

Compound A shown above, 2,2-d₂-1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one, is obtained as a brown oil in free base form in WO 2017/165843.

The development of formulations for deuterated lumateperone derivatives has proven challenging because they have not been previously disclosed to exist in solid crystal salt forms. In free base form, these compounds are either oils or oily, sticky solids, with poor solubility, not only in water but also in many organic solvents. Preparing salts of these compound has proven to be unusually difficult. Other than the tosylate salt of Compounds B to E above, no other salts of these compounds have been specifically disclosed, nor has it been shown that these compounds form stable crystalline solids.

There is thus a need for alternative stable and pharmaceutically acceptable salts, crystals and co-crystals of deuterated derivatives of lumateperone.

### BRIEF SUMMARY

In an effort to find new salts and polymorphs of the deuterated compounds of the preset disclosure, an extensive salt screen was undertaken. A variety of salts and co-crystals were attempted to be formed using 2,2-d₂-1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one (Compound A, hereinafter also referred to as “d₂-lumateperone”) in free base as the starting material. As a starting point, it was observed that lumateperone (non-deuterated) had been found to form the following salts and co-crystals: oxalate salt, cyclamate salt, 4-aminosalicylate salt, HCl salt, mono-tosylate salt, bis-tosylate salt, free base-nicotinamide co-crystal, free base-isonicotinamide co-crystal, tosylate salt-lysine co-crystal, and tosylate salt-piperazine co-crystal. Unlike the corresponding non-deuterated compound lumateperone, however, it was unexpectedly found that Compound A did not form a 4-aminosaliclylate salt, a free base nicotinamide co-crystal, or a tosylate salt-piperazine co-crystal. In addition, while an oxalate salt crystal formed, it was found to not be stable. Furthermore, a coordination complex of cyclamate was formed, but it was not a traditional salt, as the ratio of free base compound to cyclamate was about 1:10.

The initial effort was then broadened to study salt and co-crystal formation with additional acids and crystal co-formers, in a variety of solvent systems and under a variety of reaction conditions.

Following extensive screening and experimentation, the following novel salts of d₂-lumapterone were discovered, characterized, and found to be reproducible and stable: oxalate, 4-aminosalicylate, and cyclamate.

The disclosure thus provides novel hydrochloride salts, tosylate salts, free base-isonicotinamide co-crystals and tosylate salt-lysine co-crystals of deuterated derivatives of lumateperone, together with methods of making and using the same.

Further areas of applicability of the present invention will become apparent from the detailed description provided hereinafter. It should be understood that the detailed description and specific examples, while indicating preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1(a) depicts an X-ray powder diffraction pattern for d₂-lumateperone hydrochloride salt crystal, polymorph 1. FIG. 1(b) depicts an X-ray powder diffraction pattern for d₂-lumateperone hydrochloride salt crystal, polymorph 2.

FIG. 2 depicts an X-ray powder diffraction pattern for a d₂-lumateperone mono-tosylate salt crystal.

FIG. 3 depicts an X-ray powder diffraction pattern for a d₂-lumateperone bis-tosylate salt crystal.

FIG. 4 depicts an X-ray powder diffraction pattern for a d₂-lumateperone free base-isonicotinamide co-crystal.

FIG. 5 depicts an X-ray powder diffraction pattern for a d₂-lumateperone tosylate salt-lysine free base co-crystal.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### Hydrochloride Salts

In a first embodiment, the invention provides d₂-lumateperone in hydrochloride salt form (Salt 1). The invention therefore provides the following:
1.1. Salt 1 in solid form.
1.2. Salt 1 or 1.1 in crystalline form, e.g., dry crystalline form.
1.3. Salt 1.2 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous forms.
1.4. Any foregoing form of Salt 1 in crystalline form, when crystallized from a mixture of hydrochloric acid and d₂-lumateperone, e.g., in an organic solvent, e.g., comprising toluene, ethyl acetate, CPME, or mixtures thereof; e.g., wherein the hydrochloric acid and d₂-lumateperone are in a molar ratio of about 1:1, and the solvent is toluene or CPME, optionally the concentration of d₂-lumateperone is at least 150 g/L or at least 200 g/L.
1.5. Any foregoing form of Salt 1 which is a solvate, e.g., an ethyl acetate, CPME or toluene solvate.
1.6. Any foregoing form of Salt 1 which is not a solvate.
1.7. Any foregoing form of Salt 1 which is a hydrate.
1.8. Any foregoing form of Salt 1 which is not a hydrate.
1.9. Any foregoing form of Salt 1 formed by combining hydrochloric acid and d₂-lumateperone free base in about a 1:1 molar ratio.
1.10. Any foregoing form of Salt 1 wherein a DSC analysis shows (A) two endothermic events, at about 111° C. and about 290° C.; e.g. wherein a DSC/TGA analysis shows the first endothermic event at Tₒₙₛₑₜ=101.9° C., Tₚₑₐₖ=110.9° C. and ΔE=−18.4 J/g and the second at Tₒₙₛₑₜ=278.7° C., Tₚₑₐₖ=290.0° C. and ΔE=−148.6 J/g; or (B) two endothermic events, at about 108° C. and about 290° C.; e.g. wherein a DSC/TGA analysis shows the first endothermic event at Tₒₙₛₑₜ=93.6° C., Tₚₑₐₖ=108.0° C. and ΔE=−30.0 J/g and the second at Tₒₙₛₑₜ=277.5° C., Tₚₑₐₖ=289.9° C. and ΔE=−146.3 J/g.
1.11. Any foregoing form of Salt 1, in the form of a crystal having an X-ray powder diffraction pattern corresponding to the d-spacing and/or angle (2-theta) values of the following table, for example at least five, or at least six, or at least seven, or at least eight of said values, e.g., taking into account potential variations due to sample purity and instrument variation, for example 2θ shifts due to variation in X-ray wavelength, e.g., wherein the X-ray powder diffraction pattern is generated using an X-ray diffractometer with a copper anode and a nickel filter, e.g., comprising at least those peaks having a relative intensity of at least 0.4, e.g., at least 0.5, e.g., at least 0.6, e.g., comprising peaks 9, 13, 15, 22, 23, and 25 of Table (A) or peaks 6, 10, 17, 18, 20, and 21 of Table (B):

| | | | |
|---|---|---|---|
| # | Angle | d Value | Rel. Intensity |

(A) XRPD (Cu anode, Ni filter) for HCl Salt Crystal (polymorph 1)

| | | | |
|---|---|---|---|
| 1 | 5.242 | 16.84554 | 11.70% |
| 2 | 5.709 | 15.46731 | 23.70% |
| 3 | 6.39 | 13.82049 | 2.20% |
| 4 | 10.274 | 8.60283 | 9.70% |
| 5 | 10.512 | 8.40867 | 39.70% |
| 6 | 10.948 | 8.0746 | 15.50% |
| 7 | 11.678 | 7.57202 | 34.90% |
| 8 | 12.044 | 7.34265 | 53.50% |
| 9 | 12.948 | 6.83174 | 99.10% |
| 10 | 13.203 | 6.70037 | 26.00% |
| 11 | 14.084 | 6.28299 | 15.50% |
| 12 | 14.972 | 5.91243 | 37.80% |
| 13 | 15.764 | 5.61708 | 92.20% |
| 14 | 16.237 | 5.45446 | 37.00% |
| 15 | 16.501 | 5.36799 | 86.20% |
| 16 | 16.773 | 5.28161 | 22.30% |
| 17 | 17.662 | 5.01769 | 23.20% |
| 18 | 18.135 | 4.88772 | 20.70% |
| 19 | 18.649 | 4.75428 | 9.80% |
| 20 | 18.941 | 4.68146 | 23.70% |
| 21 | 19.714 | 4.49969 | 36.50% |
| 22 | 20.974 | 4.23205 | 93.20% |
| 23 | 21.595 | 4.11178 | 100.00% |
| 24 | 22.154 | 4.00926 | 70.10% |
| 25 | 23.91 | 3.71867 | 82.00% |
| 26 | 24.458 | 3.63659 | 33.00% |
| 27 | 25.059 | 3.55076 | 11.50% |
| 28 | 25.788 | 3.45193 | 10.90% |
| 29 | 26.703 | 3.33577 | 30.30% |
| 30 | 28.054 | 3.17806 | 10.60% |
| 31 | 28.883 | 3.08873 | 6.40% |
| 32 | 29.867 | 2.98912 | 7.70% |
| 33 | 30.262 | 2.95102 | 6.90% |
| 34 | 30.932 | 2.8886 | 4.10% |
| 35 | 31.483 | 2.83933 | 4.50% |
| 36 | 32.911 | 2.71932 | 4.70% |
| 37 | 33.831 | 2.6474 | 8.80% |
| 38 | 34.03 | 2.63242 | 8.00% |
| 39 | 37.892 | 2.37252 | 3.60% |
| 40 | 41.864 | 2.15613 | 3.20% |
| or | | | |

(B): XRPD (Cu anode, Ni filter) for HCI Salt Crystal (polymorph 2)

| | | | |
|---|---|---|---|
| 1 | 5.095 | 17.32919 | 26.50% |
| 2 | 5.499 | 16.0584 | 4.30% |
| 3 | 7.127 | 12.39353 | 1.80% |
| 4 | 10.432 | 8.47297 | 15.00% |
| 5 | 11.376 | 7.7719 | 35.40% |
| 6 | 11.938 | 7.40728 | 100.00% |
| 7 | 13.074 | 6.76632 | 55.90% |
| 8 | 14.411 | 6.14116 | 9.80% |
| 9 | 14.992 | 5.90451 | 57.80% |
| 10 | 16.128 | 5.49129 | 99.40% |
| 11 | 17.974 | 4.93126 | 10.20% |
| 12 | 18.547 | 4.78015 | 18.30% |
| 13 | 19.193 | 4.62066 | 18.60% |
| 14 | 19.576 | 4.53115 | 32.60% |
| 15 | 20.716 | 4.28417 | 21.80% |
| 16 | 21.134 | 4.20047 | 52.10% |
| 17 | 21.408 | 4.14722 | 64.70% |
| 18 | 22.246 | 3.99285 | 68.80% |
| 19 | 23.331 | 3.80966 | 19.80% |
| 20 | 23.977 | 3.70847 | 64.90% |
| 21 | 23.993 | 3.70596 | 64.40% |
| 22 | 24.495 | 3.63124 | 33.30% |
| 23 | 25.389 | 3.50533 | 10.10% |
| 24 | 26.589 | 3.34979 | 21.50% |
| 25 | 27.113 | 3.28615 | 18.10% |
| 26 | 27.413 | 3.2509 | 9.50% |
| 27 | 28.664 | 3.11178 | 4.70% |
| 28 | 30.464 | 2.9319 | 8.60% |
| 29 | 30.795 | 2.90113 | 7.60% |
| 30 | 31.987 | 2.79576 | 3.00% |
| 31 | 32.145 | 2.78232 | 3.00% |
| 32 | 33.114 | 2.7031 | 4.20% |
| 33 | 34.461 | 2.60047 | 4.60% |
| 34 | 39.594 | 2.27438 | 2.70% |

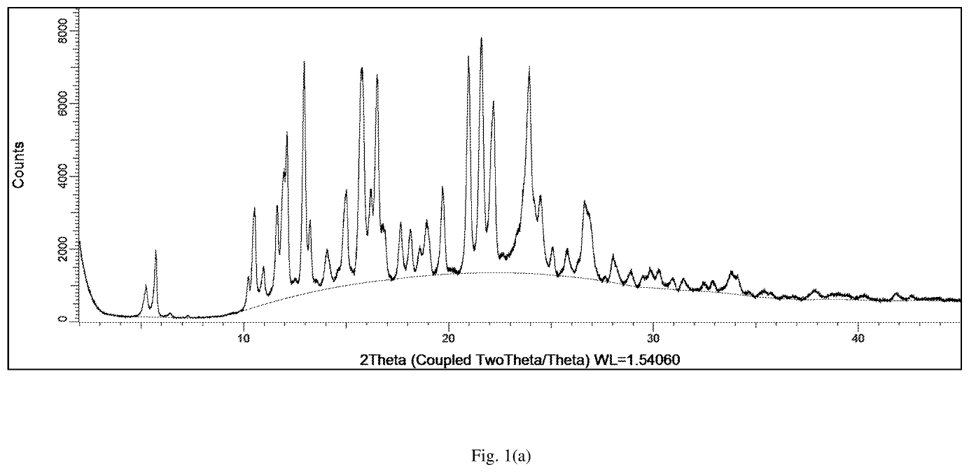

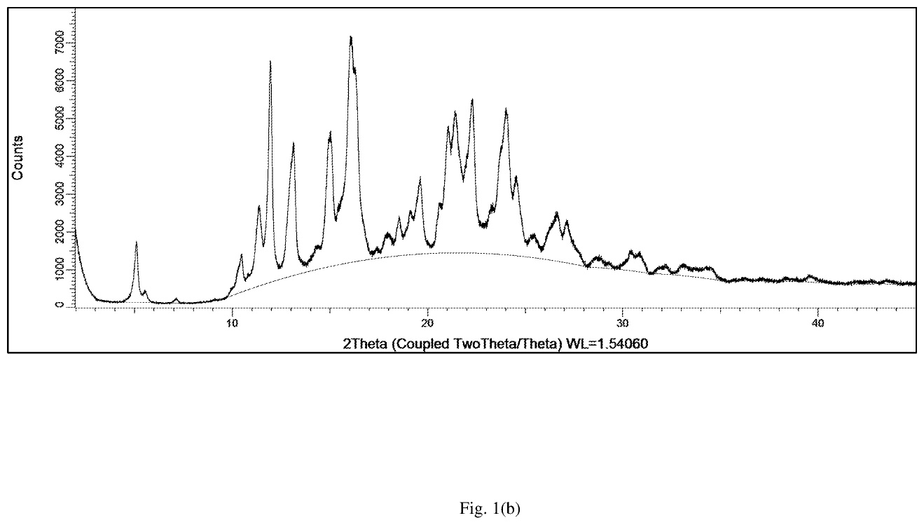

1.12. Any foregoing form of Salt 1, in the form of a crystal having an X-ray powder diffraction pattern corresponding to FIG. 1(a) or FIG. 1(b), e.g., taking into account potential variations due to sample purity and instrument variation, for example 2θ shifts due to variation in X-ray wavelength, e.g., an X-ray powder diffraction pattern corresponding to FIG. 1(a) or FIG. 1(b) generated using an X-ray diffractometer with a copper anode and a nickel filter.
1.13. Any foregoing form of Salt 1, in the form of a crystal having an X-ray powder diffraction pattern having at least 5, or at least 6, or at least 7, or at least 8, peaks having angle (2-theta) values selected from the group consisting of (A) about 5.24, 5.71, 10.51, 11.68, 12.04, 12.95, 14.98, 15.76, 16.24, 16.50, 17.66, 18.94, 19.71, 20.97, 21.60, 22.15, 23.91, and 26.70, or (B) 5.10, 10.43, 11.38, 11.94, 13.07, 14.99, 16.13, 19.58, 21.41, 22.25, 23.98, 24.50, and 26.60, about taking into account potential variations due to sample purity and instrument variation, wherein the X-ray powder diffraction pattern is generated using an X-ray diffractometer with a copper anode and a nickel filter. 1.14. Any foregoing form of Salt 1, in the form of a crystal having an X-ray powder diffraction pattern having at least 5, or at least 6, or at least 7, or at least 8, peaks having d-spacing values selected from the group consisting of (A) about 16.85, 15.47, 8.41, 7.57, 7.34, 6.83, 5.91, 5.62, 5.46, 5.37, 5.02, 4.68, 4.50, 4.23, 4.11, 4.01, 3.72, and 3.34, or (B) about 17.33, 8.48, 7.77, 7.40, 6.77, 5.90, 5.49, 4.53, 4.15, 3.99, 3.71, 3.63, and 3.35, taking into account potential variations due to sample purity and instrument variation, wherein the X-ray powder diffraction pattern is generated using an X-ray diffractometer with a copper anode and a nickel filter.
1.15. Any foregoing form of Salt 1, in the form of a crystal having an X-ray powder diffraction pattern having at least 5, or at least 6, or at least 7, or at least 8, peaks having angle (2-theta) values and/or d-spacing values as provided in group (A) of 1.13 and 1.14 or group (B) of 1.13 and 1.14.
1.16. Any foregoing form of Salt 1, in the form of a crystal having an X-ray powder diffraction powder having relative angle (2-theta) values as provided in the table (A) or (B) of embodiment 1.11, wherein the values are shifted by up to +/−0.2 degrees, e.g., wherein the values are substantially uniformly shifted by up to +/−0.2 degrees.
1.17. Any foregoing form of Salt 1, wherein the Salt has a diastereomeric excess of greater than 70%, preferably greater than 80%, more preferably greater than 90% and most preferably greater than 95%.
1.18. Any foregoing form of Salt 1, wherein the Salt has greater than natural incorporation of deuterium at the indicated deuterium positions of the structure (i.e., greater than 0.0156%).
1.19. Any foregoing form of Salt 1, wherein the Salt has substantially greater than natural incorporation of deuterium at the indicated deuterium positions of the structure (e.g., greater than 0.1%, or greater than 0.5%, or greater than 1%, or greater than 5%).
1.20. Any foregoing form of Salt 1, wherein the Salt has greater than 50% incorporation of deuterium at the indicated deuterated positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
1.21. Any foregoing form of Salt 1 exhibiting any combination of characteristics as described in 1.1-1.20.

In another embodiment, the invention provides a process (Process 1) for the production of Salt 1, comprising
(a) reacting free base d₂-lumateperone with hydrochloric acid, e.g., together with an organic solvent, e.g., comprising toluene, ethyl acetate, CPME, or mixtures thereof; e.g., wherein the hydrochloric acid and the d₂-lumateperone free base are in a molar ratio of about 1:1, and the solvent is toluene or CPME, optionally wherein the concentration of d₂-lumateperone is at least 150 g/L or at least 200 g/L; and
(b) recovering the hydrochloride salt thus formed, e.g., recovering a hydrochloride salt according to any of Salt 1, et seq. above.

In another embodiment, the invention provides a method of purifying d₂-lumateperone in free or salt form, comprising reacting a crude solution of d₂-lumateperone with hydrochloric acid, and recovering the hydrochloride salt thus formed, e.g., in accordance with Process 1, and optionally converting the hydrochloride salt back to d₂-lumateperone free base or to another salt form.

In another embodiment, the invention provides the use of hydrochloric acid in a method of isolating and/or purifying d₂-lumateperone.

In another embodiment, the invention provides a pharmaceutical composition comprising Salt 1, e.g., any of Salt 1.1-1.21, as active ingredient, in combination or association with a pharmaceutically acceptable diluent or carrier.

In another embodiment, the invention provides pharmaceutical composition comprising Salt 1, e.g., any of Salt 1.1-1.21, as active ingredient, in combination or association with a pharmaceutically acceptable diluent or carrier, wherein the Salt 1 is predominantly, or is entirely or substantially entirely, in dry crystalline form.

In a particular embodiment, the invention provides a pharmaceutical composition comprising Salt 1, e.g., any of Salt 1.1-1.21, as active ingredient, in combination or association with a pharmaceutically acceptable diluent or carrier, in the form of an injectable depot form, to provide extended release of d₂-lumateperone.

In another embodiment, the invention provides Salt 1, e.g., any of Salt 1.1-1.21, or a pharmaceutical composition comprising Salt 1, e.g., any of Salt 1.1-1.21, for use in treating a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia (e.g., Alzheimer's disease), symptoms or disorders associated with dementia (e.g., associated with Alzheimer's disease), acute anxiety, and acute depression. Particular symptoms or disorders associated with dementia (e.g., Alzheimer's disease) include anxiety, agitation, aggression, cognitive dysfunction, and memory impairment.

In another embodiment, the invention provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia, disorders associated with dementia, acute anxiety, and acute depression, comprising administering to a patient in need thereof a therapeutically effective amount of any of Salt 1, et seq.

### Mono-Tosylate Salts

In another embodiment, the invention provides d₂-lumateperone in monotosylate salt form (Salt 2). The invention therefore provides the following:

2.1. Salt 2 in solid form.
2.2. Salt 2 or 2.1 in crystalline form, e.g., dry crystalline form.
2.3. Salt 2.2 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous forms.
2.4. Any foregoing form of Salt 2 in crystalline form, when crystallized from a mixture of toluenesulfonic acid and d₂-lumateperone, e.g., in an organic solvent, e.g., comprising 2-butanone; e.g., wherein the toluenesulfonic acid and d₂-lumateperone are in a molar ratio of about 1:1, the solvent is 2-butanone, and the concentration of d₂-lumateperone is at least 50 g/L, or at least 65 g/L, or at least 100 g/L.
2.5. Any foregoing form of Salt 2 which is a solvate, e.g., a 2-butanone solvate.
2.6. Any foregoing form of Salt 2 which is not a solvate.
2.7. Any foregoing form of Salt 2 which is a hydrate.
2.8. Any foregoing form of Salt 2 which is not a hydrate.
2.9. Any foregoing form of Salt 2 formed by combining toluenesulfonic acid and d₂-lumateperone free base in about a 1:1 molar ratio.
2.10. Any foregoing form of Salt 2, wherein a DSC analysis shows one endothermic event, at about 181° C.; e.g., wherein a DSC/TGA analysis shows the endothermic event at Tₒₙₛₑₜ=179.2° C., Tₚₑₐₖ=180.9° C. and ΔE=−77.1 J/g; optionally wherein the DSC analysis further shows an exothermic event at about 277° C.; e.g., wherein a DSC/TGA analysis shows the exothermic event at Tₒₙₛₑₜ=277.1° C., Tₚₑₐₖ=285.3° C. and ΔE=+151.5 J/g.
2.11. Any foregoing form of Salt 2, in the form of a crystal having an X-ray powder diffraction pattern corresponding to the d-spacing and/or angle (2-theta) values from the following table, for example at least five, or at least six, or at least seven, or at least eight of said values, e.g., taking into account potential variations due to sample purity and instrument variation, for example 2θ shifts due to variation in X-ray wavelength, e.g., wherein the X-ray powder diffraction pattern is generated using an X-ray diffractometer with a copper anode and a nickel filter, e.g., comprising at least those peaks having a relative intensity of at least 0.3, e.g., at least 0.35, e.g., at least 0.4, e.g., comprising peaks 3, 6, 14, 19 and 28:

XRPD (Cu anode, Ni filter) for Mono-Tosylate Salt Crystal

| # | Angle | d Value | Rel. Intensity |
|---|---|---|---|
| 1 | 2.851 | 30.96656 | 17.10% |
| 2 | 5.127 | 17.2226 | 0.90% |
| 3 | 5.678 | 15.55363 | 42.70% |
| 4 | 8.513 | 10.37781 | 6.20% |
| 5 | 11.376 | 7.77174 | 19.70% |
| 6 | 12.104 | 7.30617 | 44.60% |
| 7 | 13.332 | 6.63591 | 21.30% |
| 8 | 14.211 | 6.22737 | 2.50% |
| 9 | 15.037 | 5.88696 | 3.30% |
| 10 | 15.388 | 5.75362 | 2.60% |
| 11 | 15.808 | 5.60166 | 16.60% |
| 12 | 16.041 | 5.52094 | 20.20% |
| 13 | 16.445 | 5.38617 | 13.70% |
| 14 | 17.048 | 5.19683 | 100.00% |
| 15 | 17.533 | 5.05418 | 9.00% |
| 16 | 18.173 | 4.87762 | 20.60% |
| 17 | 19.002 | 4.66654 | 19.00% |
| 18 | 19.278 | 4.60039 | 6.00% |
| 19 | 19.947 | 4.44764 | 52.60% |
| 20 | 20.763 | 4.27468 | 4.50% |
| 21 | 21.668 | 4.09818 | 10.20% |
| 22 | 21.841 | 4.06605 | 4.80% |
| 23 | 22.589 | 3.93303 | 29.30% |
| 24 | 22.815 | 3.89464 | 23.60% |
| 25 | 23.018 | 3.86072 | 14.50% |
| 26 | 23.48 | 3.78575 | 23.60% |
| 27 | 23.757 | 3.74234 | 11.10% |
| 28 | 24.314 | 3.65785 | 44.50% |
| 29 | 25.687 | 3.46536 | 5.90% |
| 30 | 25.999 | 3.42447 | 8.70% |
| 31 | 26.978 | 3.30231 | 2.80% |
| 32 | 27.264 | 3.26836 | 5.30% |
| 33 | 29.69 | 3.00656 | 3.90% |
| 34 | 30.864 | 2.89488 | 2.00% |
| 35 | 31.61 | 2.82821 | 5.10% |
| 36 | 32.314 | 2.76819 | 2.10% |
| 37 | 34.559 | 2.59334 | 3.10% |
| 38 | 34.85 | 2.5723 | 4.30% |
| 39 | 37.55 | 2.39331 | 1.60% |

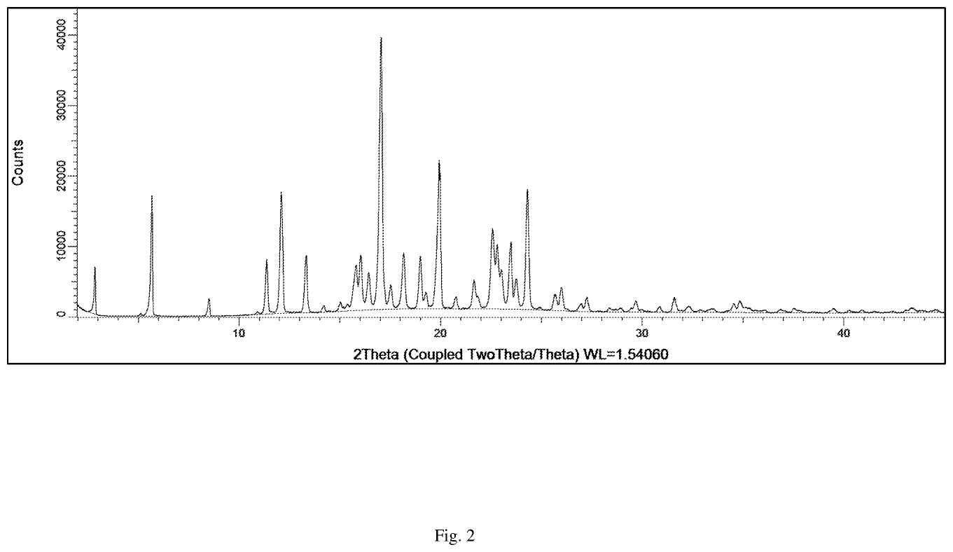

2.12. Any foregoing form of Salt 2, in the form of a crystal having an X-ray powder diffraction pattern corresponding to FIG. 2, e.g., taking into account potential variations due to sample purity and instrument variation, for example 2 θ shifts due to variation in X-ray wavelength, e.g., an X-ray powder diffraction pattern corresponding to FIG. 2 generated using an X-ray diffractometer with a copper anode and a nickel filter.
2.13. Any foregoing form of Salt 2, in the form of a crystal having an X-ray powder diffraction pattern having at least 5, or at least 6, or at least 7, or at least 8, peaks having angle (2-theta) values selected from the group consisting of about 5.68, 11.38, 12.10, 13.33, 15.81, 16.04, 16.45, 17.05, 18.17, 19.00, 19.95, 21.67, 22.59, 22.81, 23.48, and 24.31, taking into account potential variations due to sample purity and instrument variation, wherein the X-ray powder diffraction pattern is generated using an X-ray diffractometer with a copper anode and a nickel filter.
2.14. Any foregoing form of Salt 2, in the form of a crystal having an X-ray powder diffraction pattern having at least 5, or at least 6, or at least 7, or at least 8, peaks having d-spacing values selected from the group consisting of about 15.55, 7.77, 7.31, 6.64, 5.60, 5.52, 5.39, 5.20, 4.88, 4.45, 4.10, 3.93, 3.89, 3.79, and 3.66, taking into account potential variations due to sample purity and instrument variation, wherein the X-ray powder diffraction pattern is generated using an X-ray diffractometer with a copper anode and a nickel filter.
2.15. Any foregoing form of Salt 2, in the form of a crystal having an X-ray powder diffraction pattern having at least 5, or at least 6, or at least 7, or at least 8, peaks having angle (2-theta) values and/or d-spacing values as provided in 2.13 and 2.14.
2.16. Any foregoing form of Salt 2, in the form of a crystal having an X-ray powder diffraction powder having relative angle (2-theta) values as provided in the table of embodiment 2.11, wherein the values are shifted by up to +/−0.2 degrees, e.g., wherein the values are substantially uniformly shifted by up to +/−0.2 degrees.
2.17. Any foregoing form of Salt 2, wherein the Salt has a diastereomeric excess of greater than 70%, preferably greater than 80%, more preferably greater than 90% and most preferably greater than 95%.
2.18. Any foregoing form of Salt 2, wherein the Salt has greater than natural incorporation of deuterium at the indicated deuterium positions of the structure (i.e., greater than 0.0156%).
2.19. Any foregoing form of Salt 2, wherein the Salt has substantially greater than natural incorporation of deuterium at the indicated deuterium positions of the structure (e.g., greater than 0.1%, or greater than 0.5%, or greater than 1%, or greater than 5%).
2.20. Any foregoing form of Salt 2, wherein the Salt has greater than 50% incorporation of deuterium at the indicated deuterated positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
2.21. Any foregoing form of Salt 2 exhibiting any combination of characteristics as described in 2.1-2.20.

In another embodiment, the invention provides a process for the production of Salt 2 (Process 2), comprising
(a) reacting free base d₂-lumateperone with toluenesulfonic acid, e.g., together with an organic solvent, e.g., comprising 2-butanone; e.g., wherein the toluenesulfonic acid and the d₂-lumateperone free base are in a molar ratio of about 1:1, and the solvent is 2-butanone, and optionally wherein the concentration of d₂-lumateperone is at least 50 g/L, or at least 65 g/mL, or at least 100 g/L;
(b) recovering the mono-tosylate salt thus formed, e.g., recovering a mono-tosylate salt according to any of Salt 2, et seq. above.

In another embodiment, the invention provides a method of purifying d₂-lumateperone in free or salt form, comprising reacting a crude solution of d₂-lumateperone with toluenesulfonic acid, and recovering the toluenesulfonic salt thus formed, e.g., in accordance with Process 2, and optionally converting the toluenesulfonic salt back to d₂-lumateperone free base or to another salt form.

In another embodiment, the invention provides the use of toluenesulfonic acid in a method of isolating and/or purifying d₂-lumateperone, wherein the molar ratio of toluenesulfonic acid to d₂-lumateperone free base is about 1:1.

In another embodiment, the invention provides a pharmaceutical composition comprising Salt 2, e.g., any of Salt 2.1-2.21, as active ingredient, in combination or association with a pharmaceutically acceptable diluent or carrier.

In another embodiment, the invention provides pharmaceutical composition comprising Salt 2, e.g., any of Salt 2.1-2.21, as active ingredient, in combination or association with a pharmaceutically acceptable diluent or carrier, wherein the Salt 2 is predominantly, or is entirely or substantially entirely, in dry crystalline form.

In a particular embodiment, the invention provides a pharmaceutical composition comprising Salt 2, e.g., any of Salt 2.1-2.21, as active ingredient, in combination or association with a pharmaceutically acceptable diluent or carrier, in the form of an injectable depot form, to provide extended release of d₂-lumateperone.

In another embodiment, the invention provides Salt 2, e.g., any of Salt 2.1-2.21, or a pharmaceutical composition comprising Salt 2, e.g., any of Salt 2.1-2.21, for use in treating a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia (e.g., Alzheimer's disease), disorders associated with dementia (e.g., Alzheimer's disease), acute anxiety, and acute depression. Particular symptoms or disorders associated with dementia (e.g., Alzheimer's disease) include anxiety, agitation, aggression, cognitive dysfunction, and memory impairment.

In another embodiment, the invention provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia, disorders associated with dementia, acute anxiety, and acute depression, comprising administering to a patient in need thereof a therapeutically effective amount of any of Salt 2, et seq.

### Bis-Tosylate Salts

In another embodiment, the invention provides d₂-lumateperone in bis-tosylate salt form (Salt 3). The invention therefore provides the following:
3.1. Salt 3 in solid form.
3.2. Salt 3 or 3.1 in crystalline form, e.g., in dry crystalline form.
3.3. Salt 3.2 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous forms.
3.4. Any foregoing form of Salt 3 in crystalline form, when crystallized from a mixture of toluenesulfonic acid and d₂-lumateperone, e.g., in an organic solvent, e.g., comprising 2-butanone; e.g., wherein the toluenesulfonic acid and d₂-lumateperone are in a molar ratio of about 1:2, the solvent is 2-butanone, and the concentration of d₂-lumateperone is at least 50 g/L, or at least 65 g/L, or at least 100 g/L.
3.5. Any foregoing form of Salt 3 which is a solvate, e.g., a 2-butanone solvate.
3.6. Any foregoing form of Salt 3 which is not a solvate.
3.7. Any foregoing form of Salt 3 which is a hydrate.
3.8. Any foregoing form of Salt 3 which is not a hydrate.
3.9. Any foregoing form of Salt 3 formed by combining toluenesulfonic acid and d₂-lumateperone free base in a 2:1 molar ratio.
3.10. Any foregoing form of Salt 3 wherein a DSC analysis shows one endothermic event, at about 187° C.; e.g., wherein a DSC/TGA analysis shows the endothermic event at Tₒₙₛₑₜ=181.6° C., Tₚₑₐₖ=186.6° C. and ΔE=−84.4 J/g; optionally wherein the DSC analysis further shows an exothermic event at about 261° C.; e.g., wherein a DSC/TGA analysis shows the exothermic event at Tₒₙₛₑₜ=235.0° C., Tₚₑₐₖ=261.1° C. and ΔE=+331.5 J/g.
3.11. Any foregoing form of Salt 3, in the form of a crystal having an X-ray powder diffraction pattern corresponding to the d-spacing and/or angle (2-theta) values from the following table, for example at least five, or at least six, or at least seven, or at least eight of said values, e.g., taking into account potential variations due to sample purity and instrument variation, for example 2θ shifts due to variation in X-ray wavelength, e.g., wherein the X-ray powder diffraction pattern is generated using an X-ray diffractometer with a copper anode and a nickel filter, e.g., comprising at least those peaks having a relative intensity of at least 0.2, e.g., at least 0.3, e.g., at least 0.4, e.g., comprising peaks 10, 13, 19, and 20:

XRPD (Cu anode, Ni filter) for Bis-tosylate Salt Crystal

| # | Angle | d Value | Rel. Intensity |
|---|---|---|---|
| 1 | 4.146 | 21.29319 | 19.30% |
| 2 | 6.223 | 14.19209 | 1.10% |
| 3 | 8.306 | 10.63621 | 3.10% |
| 4 | 10.401 | 8.49836 | 14.30% |
| 5 | 12.209 | 7.24368 | 1.60% |
| 6 | 13.675 | 6.47004 | 7.90% |
| 7 | 14.16 | 6.24983 | 30.80% |
| 8 | 14.513 | 6.09848 | 25.00% |
| 9 | 14.905 | 5.93889 | 33.40% |
| 10 | 15.375 | 5.75839 | 43.40% |
| 11 | 15.901 | 5.56892 | 35.90% |
| 12 | 16.473 | 5.37695 | 24.20% |
| 13 | 17.093 | 5.1833 | 66.90% |
| 14 | 17.836 | 4.96894 | 13.50% |
| 15 | 17.941 | 4.94014 | 22.10% |
| 16 | 18.675 | 4.74767 | 23.10% |
| 17 | 19.193 | 4.62071 | 6.10% |
| 18 | 19.802 | 4.47986 | 6.10% |
| 19 | 20.399 | 4.35002 | 100.00% |
| 20 | 20.73 | 4.28143 | 58.50% |
| 21 | 21.301 | 4.16792 | 16.90% |
| 22 | 21.568 | 4.11697 | 11.70% |
| 23 | 22.437 | 3.95936 | 11.40% |
| 24 | 22.921 | 3.8769 | 11.60% |
| 25 | 23.198 | 3.83126 | 4.50% |
| 26 | 23.968 | 3.7098 | 22.40% |
| 27 | 24.586 | 3.61791 | 4.30% |
| 28 | 25.135 | 3.54015 | 12.60% |
| 29 | 25.895 | 3.43789 | 5.70% |
| 30 | 26.033 | 3.42 | 3.90% |
| 31 | 26.813 | 3.32231 | 6.40% |
| 32 | 27.336 | 3.25991 | 22.90% |
| 33 | 27.649 | 3.22376 | 15.80% |
| 34 | 28.582 | 3.12061 | 3.30% |
| 35 | 28.945 | 3.08226 | 4.40% |
| 36 | 29.285 | 3.04721 | 4.10% |
| 37 | 30.134 | 2.96327 | 5.80% |
| 38 | 31.612 | 2.82804 | 1.40% |
| 39 | 32.161 | 2.78099 | 2.40% |
| 40 | 33.495 | 2.67321 | 1.60% |
| 41 | 34.567 | 2.59273 | 1.30% |
| 42 | 35.452 | 2.52998 | 2.70% |
| 43 | 39.727 | 2.26707 | 2.30% |
| 44 | 42.643 | 2.11854 | 1.60% |

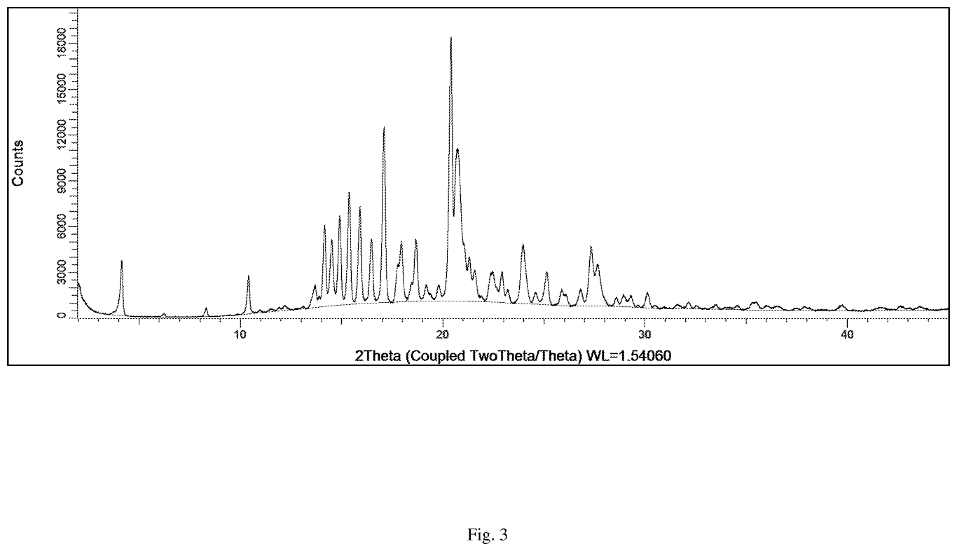

3.12. Any foregoing form of Salt 3, in the form of a crystal having an X-ray powder diffraction pattern corresponding to FIG. 3, e.g., taking into account potential variations due to sample purity and instrument variation, for example 2 Oshifts due to variation in X-ray wavelength, e.g., an X-ray powder diffraction pattern corresponding to FIG. 3 generated using an X-ray diffractometer with a copper anode and a nickel filter.
3.13. Any foregoing form of Salt 3, in the form of a crystal having an X-ray powder diffraction pattern having at least 5, or at least 6, or at least 7, or at least 8, peaks having angle (2-theta) values selected from the group consisting of about 4.15, 10.40, 14.16, 14.51, 14.91, 15.38, 15.90, 16.47, 17.10, 17.94, 18.68, 20.40, 20.73, 23.97, and 27.34, taking into account potential variations due to sample purity and instrument variation, wherein the X-ray powder diffraction pattern is generated using an X-ray diffractometer with a copper anode and a nickel filter.
3.14. Any foregoing form of Salt 3, in the form of a crystal having an X-ray powder diffraction pattern having at least 5, or at least 6, or at least 7, or at least 8, peaks having d-spacing values selected from the group consisting of about 21.29, 8.50, 6.25, 6.10, 5.94, 5.76, 5.57, 5.38, 5.18, 4.94, 4.75, 4.35, 4.28, 3.71, and 3.26, taking into account potential variations due to sample purity and instrument variation, wherein the X-ray powder diffraction pattern is generated using an X-ray diffractometer with a copper anode and a nickel filter.
3.15. Any foregoing form of Salt 3, in the form of a crystal having an X-ray powder diffraction pattern having at least 5, or at least 6, or at least 7, or at least 8, peaks having angle (2-theta) values and/or d-spacing values as provided in 3.13 and 3.14.
3.16. Any foregoing form of Salt 3, in the form of a crystal having an X-ray powder diffraction powder having relative angle (2-theta) values as provided in the table of embodiment 3.11, wherein the values are shifted by up to +/−0.2 degrees, e.g., wherein the values are substantially uniformly shifted by up to +/−0.2 degrees.
3.17. Any foregoing form of Salt 3, wherein the Salt has a diastereomeric excess of greater than 70%, preferably greater than 80%, more preferably greater than 90% and most preferably greater than 95%.
3.18. Any foregoing form of Salt 3, wherein the Salt has greater than natural incorporation of deuterium at the indicated deuterium positions of the structure (i.e., greater than 0.0156%).
3.19. Any foregoing form of Salt 3, wherein the Salt has substantially greater than natural incorporation of deuterium at the indicated deuterium positions of the structure (e.g., greater than 0.1%, or greater than 0.5%, or greater than 1%, or greater than 5%).
3.20. Any foregoing form of Salt 3, wherein the Salt has greater than 50% incorporation of deuterium at the indicated deuterated positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
3.21. Any foregoing form of Salt 3 exhibiting any combination of characteristics as described in 3.1-3.20.

In another embodiment, the invention provides a process (Process 3) for the production of Salt 3, comprising:
(a) reacting free base d₂-lumateperone with toluenesulfonic acid, e.g., together with an organic solvent, e.g., comprising 2-butanone; e.g., wherein the toluenesulfonic acid and the d₂-lumateperone free base are in a molar ratio of about 1:1 or of about 2:1, and the solvent is 2-butanone, and optionally wherein the concentration of d₂-lumateperone is at least 50 g/L, or at least 65 g/mL, or at least 100 g/L; and
(b) recovering the bis-tosylate salt thus formed, e.g., recovering a bis-tosylate salt according to any of Salt 3, et seq. above.

In another embodiment, the invention provides a method of purifying d₂-lumateperone in free or salt form, comprising reacting a crude solution of d₂-lumateperone with toluenesulfonic acid, and recovering the bis-tosylate salt thus formed, e.g., in accordance with Process 3, and optionally converting the toluenesulfonic salt back to d₂-lumateperone free base or to another salt form.

In another embodiment, the invention provides the use of toluenesulfonic acid in a method of isolating and/or purifying d₂-lumateperone, wherein the molar ratio of toluenesulfonic acid to d₂-lumateperone free base is about 1:1 or about 2:1.

In another embodiment, the invention provides a pharmaceutical composition comprising Salt 3, e.g., any of Salt 3.1-3.21, as active ingredient, in combination or association with a pharmaceutically acceptable diluent or carrier.

In another embodiment, the invention provides a pharmaceutical composition comprising Salt 3, e.g., any of Salt 3.1-3.21, as active ingredient, in combination or association with a pharmaceutically acceptable diluent or carrier, wherein the Salt 3 is predominantly, or is entirely or substantially entirely, in dry crystalline form.

In a particular embodiment, the invention provides a pharmaceutical composition comprising Salt 3, e.g., any of Salt 3.1-3.21, as active ingredient, in combination or association with a pharmaceutically acceptable diluent or carrier, in the form of an injectable depot form, to provide extended release of d₂-lumateperone.

In another embodiment, the invention provides Salt 3, e.g., any of Salt 3.1-3.21, or a pharmaceutical composition comprising Salt 3, e.g., any of Salt 3.1-3.21, for use in treating a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia (e.g., Alzheimer's disease), disorders associated with dementia (e.g., Alzheimer's disease), acute anxiety, and acute depression. Particular symptoms or disorders associated with dementia (e.g., Alzheimer's disease) include anxiety, agitation, aggression, cognitive dysfunction, and memory impairment.

In another embodiment, the invention provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia, disorders associated with dementia, acute anxiety, and acute depression, comprising administering to a patient in need thereof a therapeutically effective amount of any of Salt 3, et seq.

### Free Base-Isonicotinamide Co-Crystal

In another embodiment, the invention provides d₂-lumateperone free base in the form of a co-crystal with isonicotinamide (Co-Crystal 1). The invention therefore provides the following:
1.1. Co-Crystal 1 in solid form.
1.2. Co-Crystal 1 or 1.1 in crystalline form, e.g., in dry crystalline form.
1.3. Co-Crystal 1.2 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous forms.
1.4. Any foregoing form of Co-Crystal 1, when crystallized from a mixture of isonicotinamide and d₂-lumateperone free base, e.g., in an organic solvent, e.g., comprising methanol; e.g., wherein the isonicotinamide and d₂-lumateperone are in a molar ratio of about 1:1 or 1:2, the solvent is methanol, and the concentration of d₂-lumateperone is at least 50 g/L, or at least 100 g/L, or at least 200 g/L, and optionally wherein the Co-Crystal is crystallized from methanol.
1.5. Any foregoing form of Co-Crystal 1 which is a solvate, e.g., a methanol solvate.
1.6. Any foregoing form of Co-Crystal 1 which is not a solvate.
1.7. Any foregoing form of Co-Crystal 1 which is a hydrate.
1.8. Any foregoing form of Co-Crystal 1 which is not a hydrate.
1.9. Any foregoing form of Co-Crystal 1 formed by combining isonicotinamide and d₂-lumateperone free base in a 1:1 or 2:1 molar ratio.
1.10. Any foregoing form of Co-Crystal 1 wherein a DSC analysis shows one endothermic event, at about 151° C.; e.g., wherein a DSC/TGA analysis shows the endothermic event at Tₒₙₛₑₜ=143.2° C., Tₚₑₐₖ=150.7° C. and ΔE=−55.3 J/g.
1.11. Any foregoing form of Co-Crystal 1, in the form of a crystal having an X-ray powder diffraction pattern corresponding to the d-spacing and/or angle (2-theta) values from the following table, for example at least five, or at least six, or at least seven, or at least eight of said values, e.g., taking into account potential variations due to sample purity and instrument variation, for example 2θ shifts due to variation in X-ray wavelength, e.g., wherein the X-ray powder diffraction pattern is generated using an X-ray diffractometer with a copper anode and a nickel filter, e.g., comprising at least those peaks having a relative intensity of at least 0.5, e.g., at least 0.7, e.g., at least 0.9, e.g., comprising peak 7:

XRPD (Cu anode, Ni filter) for Free
Base-Isonicotinamide Co-Crystal

| # | Angle | d Value | Rel. Intensity |
|---|---|---|---|
| 1 | 11.358 | 7.78412 | 0.80% |
| 2 | 14.476 | 6.11379 | 1.90% |
| 3 | 18.419 | 4.81292 | 1.90% |
| 4 | 20.66 | 4.29569 | 2.80% |
| 5 | 21.928 | 4.05007 | 1.60% |
| 6 | 22.099 | 4.01919 | 2.60% |
| 7 | 22.912 | 3.87828 | 100.00% |
| 8 | 24.214 | 3.67263 | 1.90% |
| 9 | 28.509 | 3.12837 | 1.30% |
| 10 | 28.526 | 3.12655 | 1.30% |
| 11 | 29.856 | 2.99023 | 0.70% |
| 12 | 32.096 | 2.78645 | 0.90% |
| 13 | 32.121 | 2.78437 | 0.90% |
| 14 | 34.737 | 2.58043 | 0.60% |
| 15 | 34.811 | 2.57513 | 0.60% |
| 16 | 36.547 | 2.45668 | 1.30% |
| 17 | 36.572 | 2.45506 | 1.30% |

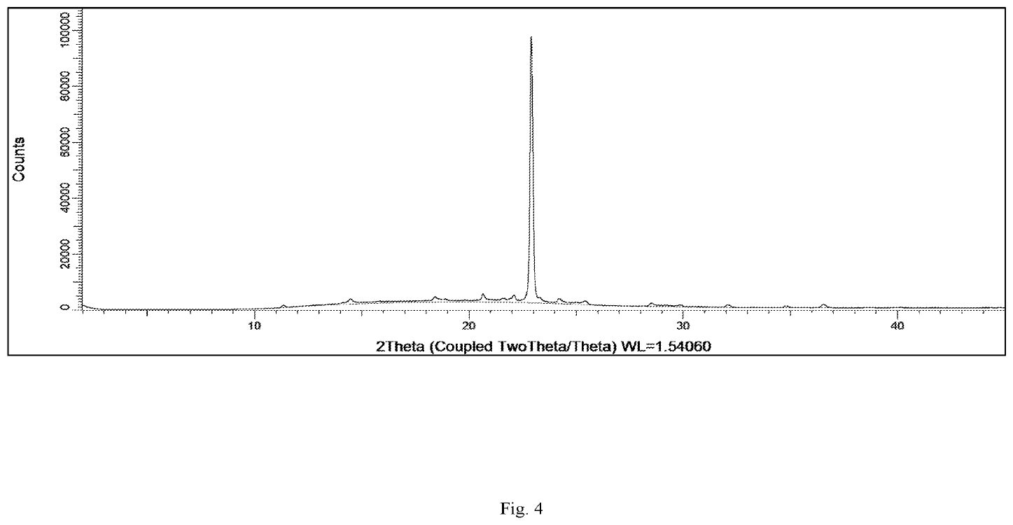

1.12. Any foregoing form of Co-Crystal 1, in the form of a crystal having an X-ray powder diffraction pattern corresponding to FIG. 4, e.g., taking into account potential variations due to sample purity and instrument variation, for example 2θ shifts due to variation in X-ray wavelength, e.g., an X-ray powder diffraction pattern corresponding to FIG. 4 generated using an X-ray diffractometer with a copper anode and a nickel filter.
1.13. Any foregoing form of Co-Crystal 1, in the form of a crystal having an X-ray powder diffraction pattern having at least a peak having an angle (2-theta) value of about 22.91, taking into account potential variations due to sample purity and instrument variation, wherein the X-ray powder diffraction pattern is generated using an X-ray diffractometer with a copper anode and a nickel filter, optionally wherein said peak is the only major peak (e.g., all other peaks have a relative intensity of less than 3.0%).
1.14. Any foregoing form of Co-Crystal 1, in the form of a crystal having an X-ray powder diffraction pattern having at least a peak having a d-spacing value of about 3.88, taking into account potential variations due to sample purity and instrument variation, wherein the X-ray powder diffraction pattern is generated using an X-ray diffractometer with a copper anode and a nickel filter, optionally wherein said peak is the only major peak (e.g., all other peaks have a relative intensity of less than 3.0%).
1.15. Any foregoing form of Co-Crystal 1, in the form of a crystal having an X-ray powder diffraction pattern having only a peak having the angle (2-theta) values and/or d-spacing value as provided in 1.13 and 1.14.
1.16. Any foregoing form of Co-Crystal 1, in the form of a crystal having an X-ray powder diffraction powder having relative angle (2-theta) values as provided in the table of embodiment 1.11, wherein the values are shifted by up to +/−0.2 degrees, e.g., wherein the values are substantially uniformly shifted by up to +/−0.2 degrees.
1.17. Any foregoing form of Co-Crystal 1, wherein the Co-Crystal has a diastereomeric excess of greater than 70%, preferably greater than 80%, more preferably greater than 90% and most preferably greater than 95%.
1.18. Any foregoing form of Co-Crystal 1, wherein the Co-Crystal has greater than natural incorporation of deuterium at the indicated deuterium positions of the structure (i.e., greater than 0.0156%).
1.19. Any foregoing form of Co-Crystal 1, wherein the Co-Crystal has substantially greater than natural incorporation of deuterium at the indicated deuterium positions of the structure (e.g., greater than 0.1%, or greater than 0.5%, or greater than 1%, or greater than 5%).
1.20. Any foregoing form of Co-Crystal 1, wherein the Co-Crystal has greater than 50% incorporation of deuterium at the indicated deuterated positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
1.21. Any foregoing form of Co-Crystal 1 exhibiting any combination of characteristics as described in 1.1-1.20.

In another embodiment, the invention provides a process (Process 4) for the production of Co-Crystal 1, comprising
(a) combining free base d₂-lumateperone with isonicotinamide, e.g., together with an organic solvent, e.g., comprising 2-butanone; e.g., wherein the isonicotinamide and the d₂-lumateperone free base are in a molar ratio of about 1:1 or 2:1, and the solvent is methanol, and optionally wherein the concentration of d₂-lumateperone is at least 50 g/L, or at least 100 g/mL, or at least 200 g/L; and
(b) removing the solvent and recovering the Co-Crystal thus formed, e.g., recovering an isonicotinamide Co-Crystal according to any of Co-Crystal 1, et seq. above.

In another embodiment, the invention provides a method of purifying d₂-lumateperone in free or salt form, comprising combining d₂-lumateperone free base with isonicotinamide, and recovering the Co-Crystal thus formed, e.g., in accordance with Process 4, and optionally converting the Co-Crystal back to d₂-lumateperone free base or to another salt form.

In another embodiment, the invention provides the use of isonicotinamide in a method of isolating and/or purifying d₂-lumateperone.

In another embodiment, the invention provides a pharmaceutical composition comprising Co-Crystal 1, e.g., any of Co-Crystal 1.1-1.21, as active ingredient, in combination or association with a pharmaceutically acceptable diluent or carrier.

In another embodiment, the invention provides a pharmaceutical composition comprising Co-Crystal 1, e.g., any of Co-Crystal 1.1-1.21, as active ingredient, in combination or association with a pharmaceutically acceptable diluent or carrier, wherein the Co-Crystal 1 is predominantly, or is entirely or substantially entirely, in dry crystalline form.

In a particular embodiment, the invention provides a pharmaceutical composition comprising Co-Crystal 1, e.g., any of Co-Crystal 1.1-1.21, as active ingredient, in combination or association with a pharmaceutically acceptable diluent or carrier, in the form of an injectable depot form, to provide extended release of d₂-lumateperone.

In another embodiment, the invention provides Co-Crystal 1, e.g., any of Co-Crystal 1.1-1.21, or a pharmaceutical composition comprising Co-Crystal 1, e.g., any of Co-Crystal 1.1-1.21, for use in treating a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia (e.g., Alzheimer's disease), disorders associated with dementia (e.g., Alzheimer's disease), acute anxiety, and acute depression. Particular symptoms or disorders associated with dementia (e.g., Alzheimer's disease) include anxiety, agitation, aggression, cognitive dysfunction, and memory impairment.

In another embodiment, the invention provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia, disorders associated with dementia, acute anxiety, and acute depression, comprising administering to a patient in need thereof a therapeutically effective amount of any of Co-Crystal 1, et seq.

### Tosylate-Lysine Co-Crystal

In another embodiment, the invention provides d₂-lumateperone tosylate in the form of a co-crystal with L-lysine free base (Co-Crystal 2). The invention therefore provides the following:
2.1. Co-Crystal 2 in solid form.
2.2. Co-Crystal 2 or 2.1 in crystalline form, e.g., in dry crystalline form.
2.3. Co-Crystal 2.2 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous forms.
2.4. Any foregoing form of Co-Crystal 2, when crystallized from a mixture of L-lysine free base and d₂-lumateperone tosylate (e.g., mono-tosylate), e.g., in an organic solvent, e.g., comprising methanol; e.g., wherein the lysine free base and d₂-lumateperone are in a molar ratio of about 1:1, the solvent is methanol, and the concentration of d₂-lumateperone is at least 50 g/L, or at least 80 g/L, and optionally wherein the Co-Crystal is crystallized from methanol.
2.5. Any foregoing form of Co-Crystal 2 which is a solvate, e.g., a methanol solvate.
2.6. Any foregoing form of Co-Crystal 2 which is not a solvate.
2.7. Any foregoing form of Co-Crystal 2 which is a hydrate.
2.8. Any foregoing form of Co-Crystal 2 which is not a hydrate.
2.9. Any foregoing form of Co-Crystal 2 formed by combining L-lysine free base and d₂-lumateperone tosylate (e.g., mono-tosylate) in a 1:1 molar ratio.
2.10. Any foregoing form of Co-Crystal 2 wherein a DSC analysis shows two endothermic events, at about 204° C. and 222° C.; e.g., wherein a DSC/TGA analysis shows a first endothermic event at Tₒₙₛₑₜ=193.1° C., Tₚₑₐₖ=203.1° C. and ΔE=−72.2 J/g, and a second endothermic event at Tₒₙₛₑₜ=188.8° C., Tₚₑₐₖ=221.7° C. and ΔE=−109.3 J/g.
2.11. Any foregoing form of Co-Crystal 2, in the form of a crystal having an X-ray powder diffraction pattern corresponding to the d-spacing and/or angle (2-theta) values from the following table, for example at least five, or at least six, or at least seven, or at least eight of said values, e.g., taking into account potential variations due to sample purity and instrument variation, for example 2θ shifts due to variation in X-ray wavelength, e.g., wherein the X-ray powder diffraction pattern is generated using an X-ray diffractometer with a copper anode and a nickel filter, e.g., comprising at least those peaks having a relative intensity of at least 0.2, e.g., at least 0.3, e.g., at least 0.4, e.g., comprising peaks 2, 11, 14, and 20:

XRPD (Cu anode, Ni filter) for Tosylate-Lysine Co-Crystal

| # | Angle | d Value | Rel. Intensity |
|---|---|---|---|
| 1 | 7.471 | 11.82319 | 5.90% |
| 2 | 9.541 | 9.26261 | 44.60% |
| 3 | 11.149 | 7.92974 | 3.30% |
| 4 | 11.522 | 7.67421 | 11.80% |
| 5 | 12.469 | 7.09293 | 8.10% |
| 6 | 14.969 | 5.9137 | 10.30% |
| 7 | 15.437 | 5.73524 | 12.70% |
| 8 | 17.543 | 5.05147 | 3.20% |
| 9 | 18.184 | 4.87469 | 22.40% |
| 10 | 19.14 | 4.63327 | 10.60% |
| 11 | 20 | 4.43594 | 100.00% |
| 12 | 20.177 | 4.39754 | 18.60% |
| 13 | 20.781 | 4.27107 | 13.90% |
| 14 | 22.386 | 3.96821 | 64.10% |
| 15 | 22.708 | 3.91277 | 21.00% |
| 16 | 23.152 | 3.83863 | 7.30% |
| 17 | 24.535 | 3.6254 | 8.30% |
| 18 | 24.857 | 3.57907 | 12.80% |
| 19 | 25.405 | 3.50311 | 2.10% |
| 20 | 25.913 | 3.43555 | 82.20% |
| 21 | 28.025 | 3.18128 | 1.40% |
| 22 | 28.91 | 3.08591 | 1.70% |
| 23 | 29.73 | 3.00261 | 7.90% |
| 24 | 30.211 | 2.95595 | 22.20% |
| 25 | 30.655 | 2.91408 | 4.30% |
| 26 | 30.901 | 2.8915 | 6.60% |
| 27 | 32.121 | 2.78434 | 4.10% |
| 28 | 33.567 | 2.66765 | 5.10% |
| 29 | 33.892 | 2.64284 | 4.70% |
| 30 | 34.195 | 2.62011 | 3.30% |
| 31 | 35.017 | 2.56045 | 2.90% |
| 32 | 35.857 | 2.50239 | 4.20% |
| 33 | 37.319 | 2.4076 | 4.70% |
| 34 | 39.287 | 2.29144 | 2.40% |
| 35 | 39.712 | 2.26788 | 1.20% |
| 36 | 40.68 | 2.2161 | 1.60% |
| 37 | 42.856 | 2.10851 | 1.00% |
| 38 | 43.764 | 2.06684 | 1.50% |

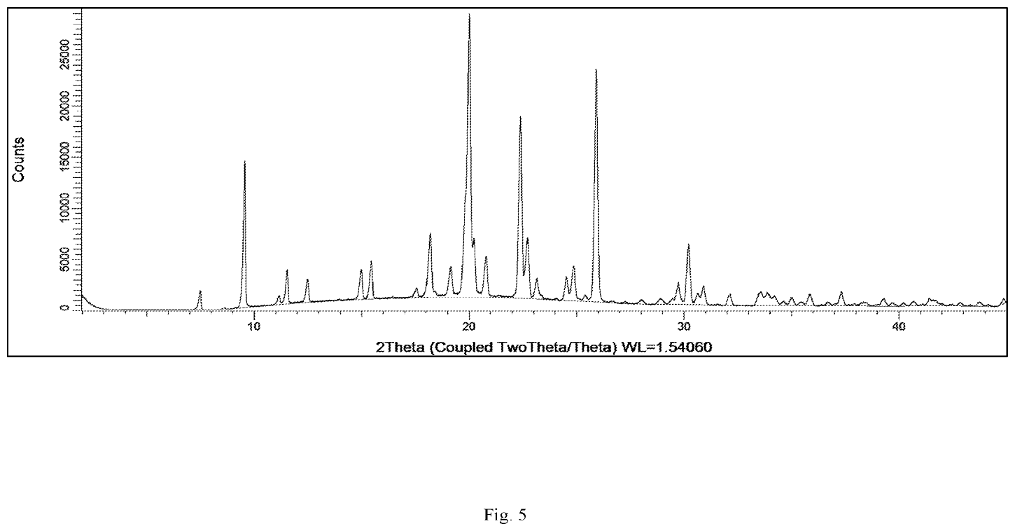

2.12. Any foregoing form of Co-Crystal 2, in the form of a crystal having an X-ray powder diffraction pattern corresponding to FIG. 5, e.g., taking into account potential variations due to sample purity and instrument variation, for example 20 shifts due to variation in X-ray wavelength, e.g., an X-ray powder diffraction pattern corresponding to FIG. 5 generated using an X-ray diffractometer with a copper anode and a nickel filter.
2.13. Any foregoing form of Co-Crystal 2, in the form of a crystal having an X-ray powder diffraction pattern having at least 5, or at least 6, or at least 7, or at least 8, peaks having angle (2-theta) values selected from the group consisting of about 9.54, 11.52, 14.97, 15.44, 18.18, 20.00, 20.18, 22.39, 22.71, 24.86, 25.91, and 30.21, taking into account potential variations due to sample purity and instrument variation, wherein the X-ray powder diffraction pattern is generated using an X-ray diffractometer with a copper anode and a nickel filter.
2.14. Any foregoing form of Co-Crystal 2, in the form of a crystal having an X-ray powder diffraction pattern having at least 5, or at least 6, or at least 7, or at least 8, peaks having d-spacing values selected from the group consisting of about 9.27, 7.67, 5.91, 5.73, 4.87, 4.44, 4.40, 3.97, 3.91, 3.58, 3.44, and 2.96, taking into account potential variations due to sample purity and instrument variation, wherein the X-ray powder diffraction pattern is generated using an X-ray diffractometer with a copper anode and a nickel filter.
2.15. Any foregoing form of Co-Crystal 2, in the form of a crystal having an X-ray powder diffraction pattern having at least 5, or at least 6, or at least 7, or at least 8, peaks having angle (2-theta) values and/or d-spacing values as provided in 2.13 and 2.14.
2.16. Any foregoing form of Co-Crystal 2, in the form of a crystal having an X-ray powder diffraction powder having relative angle (2-theta) values as provided in the table of embodiment 2.11, wherein the values are shifted by up to +/−0.2 degrees, e.g., wherein the values are substantially uniformly shifted by up to +/−0.2 degrees.
2.17. Any foregoing form of Co-Crystal 2, wherein the Co-Crystal has a diastereomeric excess of greater than 70%, preferably greater than 80%, more preferably greater than 90% and most preferably greater than 95%.
2.18. Any foregoing form of Co-Crystal 2, wherein the Co-Crystal has greater than natural incorporation of deuterium at the indicated deuterium positions of the structure (i.e., greater than 0.0156%).
2.19. Any foregoing form of Co-Crystal 2, wherein the Co-Crystal has substantially greater than natural incorporation of deuterium at the indicated deuterium positions of the structure (e.g., greater than 0.1%, or greater than 0.5%, or greater than 1%, or greater than 5%).
2.20. Any foregoing form of Co-Crystal 2, wherein the Co-Crystal has greater than 50% incorporation of deuterium at the indicated deuterated positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
2.21. Any foregoing form of Co-Crystal 2 exhibiting any combination of characteristics as described in 2.1-2.20.

In another embodiment, the invention provides a process (Process 5) for the production of Co-Crystal 2, comprising
(a) combining d₂-lumateperone tosylate (e.g., mono-tosylate) with L-lysine free base, e.g., together with an organic solvent, e.g., comprising methanol; e.g., wherein the d₂-lumateperone tosylate (e.g., mono-tosylate) and the L-lysine free base are in a molar ratio of about 1:1, and the solvent is methanol, and optionally wherein the concentration of d₂-lumateperone is at least 50 g/L, or at least 80 g/L; and
(b) removing the solvent and recovering the Co-Crystal thus formed, e.g., recovering a lysine Co-Crystal according to any of Co-Crystal 2, et seq. above.

In another embodiment, the invention provides a method of purifying d₂-lumateperone in free or salt form, comprising combining d₂-lumateperone tosylate (e.g., mono-tosylate) with L-lysine free base, and recovering the Co-Crystal thus formed, e.g., in accordance with Process 5, and optionally converting the Co-Crystal back to d₂-lumateperone free base or to another salt form.

In another embodiment, the invention provides the use of L-lysine free base in a method of isolating and/or purifying d₂-lumateperone.

In another embodiment, the invention provides a pharmaceutical composition comprising Co-Crystal 2, e.g., any of Co-Crystal 2.1-2.21, as active ingredient, in combination or association with a pharmaceutically acceptable diluent or carrier.

In another embodiment, the invention provides a pharmaceutical composition comprising Co-Crystal 2, e.g., any of Co-Crystal 2.1-2.21, as active ingredient, in combination or association with a pharmaceutically acceptable diluent or carrier, wherein the Co-Crystal 2 is predominantly, or is entirely or substantially entirely, in dry crystalline form.

In a particular embodiment, the invention provides a pharmaceutical composition comprising Co-Crystal 2, e.g., any of Co-Crystal 2.1-2.21, as active ingredient, in combination or association with a pharmaceutically acceptable diluent or carrier, in the form of an injectable depot form, to provide extended release of d₂-lumateperone.

In another embodiment, the invention provides Co-Crystal 2, e.g., any of Co-Crystal 2.1-2.21, or a pharmaceutical composition comprising Co-Crystal 2, e.g., any of Co-Crystal 2.1-2.21, for use in treating a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia (e.g., Alzheimer's disease), disorders associated with dementia (e.g., Alzheimer's disease), acute anxiety, and acute depression. Particular symptoms or disorders associated with dementia (e.g., Alzheimer's disease) include anxiety, agitation, aggression, cognitive dysfunction, and memory impairment.

In another embodiment, the invention provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia, disorders associated with dementia, acute anxiety, and acute depression, comprising administering to a patient in need thereof a therapeutically effective amount of any of Co-Crystal 2, et seq.

### Other Salts and Co-Crystals

In another embodiment, the invention provides:
(1) 1-(4-fluorophenyl)-4-((6bR,10aS)-3-(d₃-methyl)-2,3,6b,7,10,10a-hexahydro-1H-pyrido[3′,4′:4,5]pyrrolo[1,2,3-de]quinoxalin-8(9H)-yl)butan-1-one (d₃-lumateperone),
(2) 2-2-d₂-1-(4-fluorophenyl)-4-((6bR,10aS)-3-(d₃-methyl)-2,3,6b,7,10,10a-hexahydro-1H-pyrido[3′,4′:4,5]pyrrolo[1,2,3-de]quinoxalin-8(9H)-yl)butan-1-one (d₅-lumateperone),
(3) 1,1,2,2-d₄-1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one (d₄-lumateperone), or
(4) 1,1,2,2-d₄-1-(4-fluorophenyl)-4-((6bR,10aS)-3-(d₃-methyl)-2,3,6b,7,10,10a-hexahydro-1H-pyrido[3′,4′:4,5]pyrrolo[1,2,3-de]quinoxalin-8(9H)-yl)butan-1-one (d₇-lumateperone), in the form of a stable salt or co-crystal (Salt or Co-Crystal 4). The invention further provides the following:
   4.1. Salt or Co-Crystal 4 in solid form.
   4.2. Salt or Co-Crystal 4 in crystalline form, e.g., in dry crystalline form.
   4.3. Salt or Co-Crystal 4 in a homogeneous crystal form, free or substantially free of other forms, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous forms.
   4.4. Salt or Co-Crystal 4, wherein the Salt or Co-Crystal is a salt selected from the group consisting of hydrochloride salt, mono-tosylate salt, and bis-tosylate salt;
   4.5. Salt or Co-Crystal 4.4, when crystallized from a mixture of hydrochloride acid, or toluenesulfonic acid and d₃-lumateperone, d₅-lumateperone, d₄-lumateperone, or d₇-lumateperone, e.g., in an organic solvent, e.g., comprising methanol, ethyl acetate, toluene, CPME, or 2-butanone; e.g., wherein the acid and deuterated lumateperone are in a molar ratio of about 1:1 or 1:2, and the concentration of d₂-lumateperone is at least 50 g/L, or at least 65 g/L, or at least 100 g/L, or at least 200 g/L.
   4.6. Salt or Co-Crystal 4, wherein the Salt or Co-Crystal is a free base-isonicotinamide co-crystal or a tosylate salt-lysine co-crystal.
   4.7. Salt or Co-Crystal 4.6, when crystallized from a mixture of lysine free base and d₃-lumateperone, d₅-lumateperone, d₄-lumateperone, or d₇-lumateperone, free base or tosylate (e.g., mono-tosylate), e.g. in an organic solvent, e.g., comprising methanol; e.g., wherein the lysine free base or isonicotinamide and the deuterated lumateperone are in a molar ratio of about 1:1, the solvent is methanol, and the concentration of the deuterated lumateperone is at least 50 g/L, or at least 65 g/L, or at least 100 g/L, or at least 200 g/L.
   4.8. Any foregoing form of Salt or Co-Crystal 4 which is a solvate.
   4.9. Any foregoing form of Salt or Co-Crystal 4 which is not a solvate.
   4.10. Any foregoing form of Salt or Co-Crystal 4 which is a hydrate.
   4.11. Any foregoing form of Salt or Co-Crystal 4 which is not a hydrate.
   4.12. Any foregoing form of Salt or Co-Crystal 4, wherein the Salt or Co-Crystal has a diastereomeric excess of greater than 70%, preferably greater than 80%, more preferably greater than 90% and most preferably greater than 95%.
   4.13. Any foregoing form of Salt or Co-Crystal 4, wherein the Salt or Co-Crystal has greater than natural incorporation of deuterium at the indicated deuterium positions of the structure (i.e., greater than 0.0156%).
   4.14. Any foregoing form of Salt or Co-Crystal 4, wherein the Salt or Co-Crystal has substantially greater than natural incorporation of deuterium at the indicated deuterium positions of the structure (e.g., greater than 0.1%, or greater than 0.5%, or greater than 1%, or greater than 5%).
   4.15. Any foregoing form of Salt or Co-Crystal 4, wherein the Salt or Co-Crystal has greater than 50% incorporation of deuterium at the indicated deuterated positions of the structure (i.e., greater than 50 atom % D), e.g., greater than 60%, or greater than 70%, or greater than 80%, or greater than 90% or greater than 95%, or greater than 96%, or greater than 97%, or greater than 98%, or greater than 99%.
   4.16. Any foregoing form of Salt or Co-Crystal 4 exhibiting any combination of characteristics as described in 4.1-4.15.

In another embodiment, the invention provides a process (Process 6) for the production of Salt or Co-Crystal 4, comprising
(a) reacting d₃-lumateperone, d₅-lumateperone, d₄-lumateperone, or d₇-lumateperone, free base or tosylate (e.g., mono-tosylate), with hydrochloric acid, toluenesulfonic acid, lysine free base (e.g., L-lysine), or isonicotinamide, e.g., together with an organic solvent, e.g., comprising methanol, ethyl acetate, CPME, toluene, or 2-butanone; e.g., wherein the acid or the co-former and the deuterated lumateperone in a molar ratio of about 1:1 or 2:1, and optionally wherein the concentration of the deuterated lumateperone is at least 50 g/L, or at least 65 g/L, or at least 100 g/L, or at least 200 g/L; and
(b) recovering the salt or co-crystal thus formed, e.g., recovering Salt or Co-Crystal 4 or any of 4.1-4.15.

In another embodiment, the invention provides a method of purifying d₃-lumateperone, d₅-lumateperone, d₄-lumateperone, or d₇-lumateperone, in free or salt form, comprising reacting a crude solution of the deuterated lumateperone with hydrochloric acid, toluenesulfonic acid, lysine free base (e.g., L-lysine), or isonicotinamide, and recovering the salt or co-crystal thus formed, e.g., in accordance with Process 6, and optionally converting the salt or co-crystal back to d₃-lumateperone, d₅-lumateperone, d₄-lumateperone, or d₇-lumateperone free base or to another salt form.

In another embodiment, the invention provides the use of hydrochloric acid, toluenesulfonic acid, lysine free base (e.g., L-lysine), or isonicotinamide, in a method of isolating and/or purifying d₃-lumateperone, d₅-lumateperone, d₄-lumateperone, or d₇-lumateperone, wherein the molar ratio of the acid or co-former to the deuterated lumateperone free base or salt is about 1:1 or 2:1.

In another embodiment, the invention provides a pharmaceutical composition comprising Salt or Co-Crystal 4 or any of 4.1-4.15, as active ingredient, in combination or association with a pharmaceutically acceptable diluent or carrier.

In another embodiment, the invention provides a pharmaceutical composition comprising Salt or Co-Crystal 4 or any of 4.1-4.15, as active ingredient, in combination or association with a pharmaceutically acceptable diluent or carrier, wherein the Salt or Co-Crystal 4 is predominantly, or is entirely or substantially entirely, in dry crystalline form.

In a particular embodiment, the invention provides a pharmaceutical composition comprising Salt or Co-Crystal 4 or any of 4.1-4.15, as active ingredient, in combination or association with a pharmaceutically acceptable diluent or carrier, in the form of an injectable depot form, to provide extended release of the deuterated lumateperone.

In another embodiment, the invention provides Salt or Co-Crystal 4 or any of 4.1-4.15, or a pharmaceutical composition comprising Salt or Co-Crystal 4 or any of 4.1-4.15, for use in treating a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia (e.g., Alzheimer's disease), disorders associated with dementia (e.g., Alzheimer's disease), acute anxiety, and acute depression. Particular symptoms or disorders associated with dementia (e.g., Alzheimer's disease) include anxiety, agitation, aggression, cognitive dysfunction, and memory impairment.

In another embodiment, the invention provides a method for the prophylaxis or treatment of a human suffering from a disease or abnormal condition involving or mediated by the 5-HT_{2A} receptor, serotonin transporter (SERT), and/or dopamine D₁/D₂ receptor signaling pathways, e.g., a disorder selected from obesity, anorexia, bulimia, depression (such as bipolar depression or major depressive disorder), anxiety, psychosis, schizophrenia (especially the residual symptoms and/or negative symptoms of schizophrenia), migraine, obsessive-compulsive disorder, sexual disorders, attention deficit disorder, attention deficit hyperactivity disorder, sleep disorders, conditions associated with cephalic pain, social phobias, dementia, disorders associated with dementia, acute anxiety, and acute depression, comprising administering to a patient in need thereof a therapeutically effective amount of any of Salt or Co-Crystal 4 or any of 4.1-4.15, et seq.

All salts and co-crystals described herein are preferably stable, meaning that they retain physical and chemical identity (as shown by, e.g., 1H-NMR, LCMS) over a period of at least 1 month, e.g., at least 3 months, or at least 6 months, or at least 9 months.

### EXAMPLES

The following equipment and methods are used to isolate and characterize the exemplified salt forms:

X-ray powder diffraction (XRPD): The X-ray powder diffraction studies are performed using a Bruker AXS D2 PHASER in Bragg-Brentano configuration, equipment #1549/#2353. The equipment uses a Cu anode at 30 kV, 10 mA; sample stage standard rotating; monochromatization by a Kβ-filter (0.5% Ni). Slits: fixed divergence slits 1.0 mm)(=0.61°, primary axial Soller slit 2.5°, secondary axial Soller slit 2.5°. Detector: Linear detector LYNXEYE with receiving slit 5° detector opening. The standard sample holder (0.1 mm cavity in (510) silicon wafer) has a minimal contribution to the background signal. Measurement conditions: scan range 5-45° 20, sample rotation 5 rpm, 0.5 s/step, 0.010°/step, 3.0 mm detector slit; and all measuring conditions are logged in the instrument control file. As system suitability, corundum sample A26-B26-S (NIST standard) is measured daily. The software used for data collection is Diffrac.Commander v2.0.26. Data analysis is done using Diffrac.Eva v1.4. No background correction or smoothing is applied to the patterns.

Simultaneous thermogravimetry (TGA) and differential scanning calorimetry (DSC) or TGA/DSC analysis: The TGA/DSC studies are performed using a Mettler Toledo TGA/DSC1 Stare System, equipment #1547, auto-sampler equipped, using pin-holed Al-crucibles of 40 μl. Measurement conditions: 5 min 30.0° C., 30.0-350.0° C. with 10° C./min., N2 flow of 40 ml/min. The software used for instrument control and data analysis is STARe v12.10.

Differential scanning calorimetry (DSC): The DSC studies are performed using a Mettler Toledo DSC1 STARe System, equipment #1564. The samples are made using Al crucibles (40 μl; pierced). Typically, 1-8 mg of sample is loaded onto a pre-weighed Al crucible and is kept at 30° C. for 5 minutes, after which it is heated at 10° C./min from 30° C. to 350° C. and kept at 350° C. for 1 minute. A nitrogen purge of 40 ml/min is maintained over the sample. As system suitability check Indium and Zinc are used as references. The software used for data collection and evaluation is STARe Software v12.10 build 5937. No corrections are applied to the thermogram.

Fourier transform infrared spectroscopy (FT-IR): The FT-IR studies are performed using a Thermo Scientific Nicolet iS50, equipment #2357. An attenuated total reflectance (ATR) technique was used with a beam splitter of KBr. Experiment setup of the collected sample is used number of scans 16 with a resolution of 4 from 400 cm-1 to 4000 cm-1. The software OMNIC version 9.2 is used for data collection and evaluation.

Thermogravimetric analysis (TGA) with infrared spectroscopy (TGA-IR): In TGA-IR, the off-gassing materials are directed through a transfer line to a gas cell, where the infrared light interacts with the gases. The temperature ramp and first derivative weight loss information from the TGA is shown as a Gram-Schmidt (GS) profile; the GS profile essentially shows the total change in the IR signal relative to the initial state. In most cases, the GS and the derivative weight loss will be similar in shape, although the intensity of the two can differ. For this experiment are two devices coupled to each other. The TGA studies are performed using a Mettler Toledo TGA/DSC1 STARe System with a 34-position auto sampler, equipment #1547. The samples are made using Al crucibles (100 μl; pierced). Typically, 20-50 mg of sample is loaded into a pre-weighed Al crucible and is kept at 30° C. for 5 minutes after which it is heated at 10° C./min from 30° C. to 350° C. A nitrogen purge of 40 ml/min is maintained over the sample. The TGA-IR module of the Nicolet iS50 is coupled to the TGA/DSC1. The IR studies were performed using a Thermo Scientific Nicolet iS50, equipment #2357. Experiment setup of the collected series, the profile Gram-Schmidt is used number of scans 10 with a resolution of 4. The software OMNIC version 9.2 is used for data collection and evaluation.

High performance liquid chromatography (HPLC): The high performance liquid chromatography analyses are performed on LC-31, equipped with an Agilent 1100 series G1322A degasser equipment #1894, an Agilent 1100 series G1311A quaternary pump equipment #1895, an Agilent 1100 series G1313A ALS equipment #1896, an Agilent 1100 series G1318A column equipment #1897 and an Agilent 1100 series G1314A VWD equipment #1898/LC-34, equipped with an Agilent 1200 series G1379B degasser equipment #2254, an Agilent 1100 series G1311A quaternary pump equipment #2255, Agilent 1100 series G1367A WPALS equipment #1656, an Agilent 1100 series G1316A column equipment #2257 and an Agilent 1100 series G1315B DAD equipment #2258. Data is collected and evaluated using Agilent ChemStation for LC systems Rev. B.04.02[96]. Solutions are prepared as follows: Mobile phase A: Add 800 ml of MilliQ water to a 1L volumetric flask. Add 1 ml of TFA and homogenize. Fill up to the mark with MilliQ; Mobile phase B: Add 800 ml of Acetonitrile to a 1L volumetric flask. Add 1 ml of TFA and homogenize. Fill up to the mark with Acetonitrile; Diluent: 50/50 MeOH/ACN.

During previous studies, the salt crystals and co-crystals formed by lumateperone (non-deuterated) were found to include only oxalate salt, cyclamate salt, 4-aminosalicylate salt, HCl salt (different forms), mono-tosylate salt, bis-tosylate salt, free base-nicotinamide co-crystal, free base-isonicotinamide co-crystal, tosylate-lysine co-crystal, and tosylate-piperazine co-crystal. The salt crystals were identified after extensive experimentation conducted using a salt screen with 90 different counter ions, various ratios between lumateperone free base and the selected acid, six different solvents, and including four different crystallization methods (slurry experiments, cooling crystallization, evaporation and precipitation experiments). The lumateperone co-crystals were similarly identified after extensive experimentation involving 26 candidate co-formers (including sugar alcohols, amino acids, and other compounds identified as having potential to for co-crystals, various ratios between the lumateperone and the co-former, various solvent, and three different experimental conditions (adding solutions stepwise, slurry experiments and cooling crystallization experiments).

It was therefore expected that deuterated lumateperone would be similarly difficult to form crystalline salts and co-crystals from. Unlike lumateperone, d₂-lumateperone failed to form a stable and reproducible salt with oxalic acid. Instead, it was found that an oxalate salt forms in very poor yield and over time (<3 months) it changes from a yellow powder to a sticky brown solid. XRPD confirmed that while the yellow powder was a crystalline oxalate salt, the sticky brown solid was not. Similar experiments using cyclamic acid produced green and yellow powders in very low yield, analysis of which did not suggest formation of a d₂-lumateperone cyclamate salt. Analysis suggested the formation of coordination complexes with a ratio of free base to cyclamate of about 1:10. Attempts to form a crystalline 4-aminosalicylate salt also failed, as either no solid material was obtained or only amorphous material was obtained. Attempts to form a free base-nicotinamide co-crystal or a tosylate-piperazine co-crystal failed as well. XRPD of the solids obtained in the former experiments showed only nicotinamide. The powder obtained from the latter attempt was a crystal by XRPD, but NMR indicated something have a 1:20 ratio of tosylate salt to piperazine.

Thus, it was unexpected that d₂-lumateperone would not form the same crystalline salts and co-crystals as lumateperone did. Due to these unexpected difference, additional salt and co-crystal screening was performed with d₂-lumateperone.

### Example 1: Hydrochloride Salt Crystals

Hydrogen chloride in CPME solvent was added to a solution of d₂-lumateperone free base in ethyl acetate, toluene or CPME, at a 1:1 molar ratio of free base to HCl. Then the solution was heated to 50° C., kept at this temperature for an hour and cooled to room temperature. The HCl formation experiments are summarized in the table below:

| | | | | TGA | FT-IR | FT-IR | | |
|---|---|---|---|---|---|---|---|---|
| | | | DSC | mass | correlation | correlation | LC | |
| | | | Tₚₑₐₖ | loss | with FB | with CI | purity | |
| Expt. # | Appearance | XRPD | (° C.) | (%) | (%) | (%) | (area-%) | ¹H-NMR |
| 1-1 | Clear | N/A | N/A | N/A | N/A | N/A | N/A | N/A |
| | solution | | | | | | | |
| 1-2 | Clear | N/A | N/A | N/A | N/A | N/A | N/A | N/A |
| | solution | | | | | | | |
| 1-3 | Yellowish | N/A | N/A | N/A | N/A | N/A | N/A | N/A |
| | suspension | | | | | | | |
| 1-4 | Clear | N/A | N/A | N/A | N/A | N/A | N/A | N/A |
| | solution | | | | | | | |
| 1-5 | Off-white | Crystal | N/A | N/A | N/A | N/A | N/A | N/A |
| | solid | | | | | | | |
| 1-6 | Off-white | Crystal | N/A | N/A | N/A | N/A | N/A | N/A |
| | solid | | | | | | | |
| 1-7 | Yellowish | Crystal | 110.9 | 2.4 | 60 | 6 | 98 | Shifts; |
| | powder | | | | | | | salt |
| | | | | | | | | formation |
| 1-8 | Off-white | Crystal | 108.0 | 2.6 | 59 | 7 | 98 | Shifts; |
| | powder | | | | | | | salt |
| | | | | | | | | formation |

Experiments 1-1, 1-2 and 1-3 were performed at 100 mg scale with a d₂-lumateperone free base concentration of 100 mg/mL. A clear solution was obtained for experiment 1-1 (ethyl acetate) and 1-2 (toluene), and a yellowish suspension was formed of experiment 1-3 (CPME), but the yield of solid was very low. Therefore, in experiments 1-4, 1-5 and 1-6, a 100 mg scale was used, with the concentration of d₂-lumateperone free base increased to 200 mg/mL. Experiment 1-4 (ethyl acetate) still produced as a clear solution, and experiment 1-5 (toluene) and 1-6 (CPME) both showed a yellowish slurry upon cooling and an off-white powder upon isolation. Both solids showed suggestive crystalline patterns. These two experiments were then repeated at a 600 mg scale with a concentration of 200 mg/mL, as Experiments 1-7 (toluene) and 1-8 (CPME). Two new crystalline patterns were observed which were not the same as that seen in Experiments 1-5 and 1-6. Analysis of the TGA data showed that 2.4 wt-% and 2.6 wt-% solvent residual were recorded respectively, potentially accounting for the difference in crystalline pattern.

The XRPD patterns for the salts obtained in Experiments 1-7 and 1-8 are shown in FIGS. 1(a) and 1(b), respectively. The peaks are identified in tabular form in the tables below:

TABLE 1
(A). XRPD (Cu anode, Ni filter) for
HCl Salt Crystal (polymorph 1)

| # | Angle | d Value | Rel. Intensity |
|---|---|---|---|
| 1 | 5.242 | 16.84554 | 11.70% |
| 2 | 5.709 | 15.46731 | 23.70% |
| 3 | 6.39 | 13.82049 | 2.20% |
| 4 | 10.274 | 8.60283 | 9.70% |
| 5 | 10.512 | 8.40867 | 39.70% |
| 6 | 10.948 | 8.0746 | 15.50% |
| 7 | 11.678 | 7.57202 | 34.90% |
| 8 | 12.044 | 7.34265 | 53.50% |
| 9 | 12.948 | 6.83174 | 99.10% |
| 10 | 13.203 | 6.70037 | 26.00% |
| 11 | 14.084 | 6.28299 | 15.50% |
| 12 | 14.972 | 5.91243 | 37.80% |
| 13 | 15.764 | 5.61708 | 92.20% |
| 14 | 16.237 | 5.45446 | 37.00% |
| 15 | 16.501 | 5.36799 | 86.20% |
| 16 | 16.773 | 5.28161 | 22.30% |
| 17 | 17.662 | 5.01769 | 23.20% |
| 18 | 18.135 | 4.88772 | 20.70% |
| 19 | 18.649 | 4.75428 | 9.80% |
| 20 | 18.941 | 4.68146 | 23.70% |
| 21 | 19.714 | 4.49969 | 36.50% |
| 22 | 20.974 | 4.23205 | 93.20% |
| 23 | 21.595 | 4.11178 | 100.00% |
| 24 | 22.154 | 4.00926 | 70.10% |
| 25 | 23.91 | 3.71867 | 82.00% |
| 26 | 24.458 | 3.63659 | 33.00% |
| 27 | 25.059 | 3.55076 | 11.50% |
| 28 | 25.788 | 3.45193 | 10.90% |
| 29 | 26.703 | 3.33577 | 30.30% |
| 30 | 28.054 | 3.17806 | 10.60% |
| 31 | 28.883 | 3.08873 | 6.40% |
| 32 | 29.867 | 2.98912 | 7.70% |
| 33 | 30.262 | 2.95102 | 6.90% |
| 34 | 30.932 | 2.8886 | 4.10% |
| 35 | 31.483 | 2.83933 | 4.50% |
| 36 | 32.911 | 2.71932 | 4.70% |
| 37 | 33.831 | 2.6474 | 8.80% |
| 38 | 34.03 | 2.63242 | 8.00% |
| 39 | 37.892 | 2.37252 | 3.60% |
| 40 | 41.864 | 2.15613 | 3.20% |

TABLE 1
(B). XRPD (Cu anode, Ni filter) for
HCl Salt Crystal (polymorph 2)

| # | Angle | d Value | Rel. Intensity |
|---|---|---|---|
| 1 | 5.095 | 17.32919 | 26.50% |
| 2 | 5.499 | 16.0584 | 4.30% |
| 3 | 7.127 | 12.39353 | 1.80% |
| 4 | 10.432 | 8.47297 | 15.00% |
| 5 | 11.376 | 7.7719 | 35.40% |
| 6 | 11.938 | 7.40728 | 100.00% |
| 7 | 13.074 | 6.76632 | 55.90% |
| 8 | 14.411 | 6.14116 | 9.80% |
| 9 | 14.992 | 5.90451 | 57.80% |
| 10 | 16.128 | 5.49129 | 99.40% |
| 11 | 17.974 | 4.93126 | 10.20% |
| 12 | 18.547 | 4.78015 | 18.30% |
| 13 | 19.193 | 4.62066 | 18.60% |
| 14 | 19.576 | 4.53115 | 32.60% |
| 15 | 20.716 | 4.28417 | 21.80% |
| 16 | 21.134 | 4.20047 | 52.10% |
| 17 | 21.408 | 4.14722 | 64.70% |
| 18 | 22.246 | 3.99285 | 68.80% |
| 19 | 23.331 | 3.80966 | 19.80% |
| 20 | 23.977 | 3.70847 | 64.90% |
| 21 | 23.993 | 3.70596 | 64.40% |
| 22 | 24.495 | 3.63124 | 33.30% |
| 23 | 25.389 | 3.50533 | 10.10% |
| 24 | 26.589 | 3.34979 | 21.50% |
| 25 | 27.113 | 3.28615 | 18.10% |
| 26 | 27.413 | 3.2509 | 9.50% |
| 27 | 28.664 | 3.11178 | 4.70% |
| 28 | 30.464 | 2.9319 | 8.60% |
| 29 | 30.795 | 2.90113 | 7.60% |
| 30 | 31.987 | 2.79576 | 3.00% |
| 31 | 32.145 | 2.78232 | 3.00% |
| 32 | 33.114 | 2.7031 | 4.20% |
| 33 | 34.461 | 2.60047 | 4.60% |
| 34 | 39.594 | 2.27438 | 2.70% |

The hydrochloride salts are also analyzed by DSC/TGA, HPLC, ¹H-NMR and FT-IR. DSC/TGA analysis of polymorph 1 (Exp. 1-7) shows a first endothermic event at Tₒₙₛₑₜ=101.9° C., Tₚₑₐₖ=110.9° C. and ΔE=−18.4 J/g and a second at Tₒₙₛₑₜ=278.7° C., Tₚₑₐₖ=290.0° C. and ΔE=−148.6 J/g. TGA indicates a 2.4 wt % solvent residual.

DSC/TGA analysis of polymorph 2 (Exp. 1-8) shows a first endothermic event at Tₒₙₛₑₜ=93.6° C., Tₚₑₐₖ=108.0° C. and ΔE=−30.0 J/g and a second at Tₒₙₛₑₜ=277.5° C., Tₚₑₐₖ=289.9° C. and ΔE=−146.3 J/g. TGA indicates a 2.6 wt % solvent residual.

Analysis of the HPLC data shows a purity of 98-area % for both polymorphs. Analysis of the ¹H-NMR data shows some shifts compared to the free base, which confirms the salt formation in both cases. FT-IR analysis confirms the chemical structure.

### Example 2: Mono-Tosylate Salt Crystal

p-Toluenesulfonic acid was added in a 1:1 molar ratio to a solution of d₂-lumateperone free base in 2-butanone. The solution was heated to 50° C., and kept at this temperature for 1 hour, then cooled to room temperature. Scale up experiments were analyzed by XRPD, TGA, DSC, FT-IR, LC and 1H-NMR. The results are summarized in the table below:

| | | | | TGA | FT-IR | FT-IR | | |
|---|---|---|---|---|---|---|---|---|
| | | | DSC | mass | correlation | correlation | LC | |
| | | | Tₚₑₐₖ | loss | with FB | with CI | purity | |
| Expt. # | Appearance | XRPD | (° C.) | (%) | (%) | (%) | (area-%) | ¹H-NMR |
| 2-1 | Greenish | Crystal | N/A | N/A | N/A | N/A | N/A | N/A |
| | solid | | | | | | | |
| 2-2 | Off-white | Crystal | 180.9 | <0.1 | 28 | 15 | 90.9 | Ratio 1:1 |
| | powder | | | | | | | |
| 2-3 | Off-white | Crystal | N/A | N/A | N/A | N/A | N/A | N/A |
| | powder | | | | | | | |

Experiment 2-1 was performed at 100 mg scale (100 mg/mL), and a greenish slurry was obtained which was isolated as a greenish solid. XRPD showed mono-tosylate salt formation (spectrum not shown). Scale up of this experiment was performed at 1000 mg scale (66.7 mg/mL) (Exp. 2-2). The product was isolated as an off-white powder. Salt formation is confirmed by 1H-NMR. An additional experiment (Exp. 2-3) was performed at 2000 mg scale with a concentration of 50 mg/mL. This material is used for the tosylate co-crystal formation experiments. The mixture of d₂-lumateperone free base, p-toluenesulfonic acid and 2-butanone was heated to 50° C. for 1 hour, then cooled to room temperature and precipitation occurred after 3 hours. The product was isolated as an off-white powder. The XRPD from Exp. 2-3 is shown in FIG. 2.

The peak list corresponding to FIG. 2 is shown below in tabular form:

TABLE 2
XRPD (Cu anode, Ni filter) for Mono-Tosylate Salt Crystal

| # | Angle | d Value | Rel. Intensity |
|---|---|---|---|
| 1 | 2.851 | 30.96656 | 17.10% |
| 2 | 5.127 | 17.2226 | 0.90% |
| 3 | 5.678 | 15.55363 | 42.70% |
| 4 | 8.513 | 10.37781 | 6.20% |
| 5 | 11.376 | 7.77174 | 19.70% |
| 6 | 12.104 | 7.30617 | 44.60% |
| 7 | 13.332 | 6.63591 | 21.30% |
| 8 | 14.211 | 6.22737 | 2.50% |
| 9 | 15.037 | 5.88696 | 3.30% |
| 10 | 15.388 | 5.75362 | 2.60% |
| 11 | 15.808 | 5.60166 | 16.60% |
| 12 | 16.041 | 5.52094 | 20.20% |
| 13 | 16.445 | 5.38617 | 13.70% |
| 14 | 17.048 | 5.19683 | 100.00% |
| 15 | 17.533 | 5.05418 | 9.00% |
| 16 | 18.173 | 4.87762 | 20.60% |
| 17 | 19.002 | 4.66654 | 19.00% |
| 18 | 19.278 | 4.60039 | 6.00% |
| 19 | 19.947 | 4.44764 | 52.60% |
| 20 | 20.763 | 4.27468 | 4.50% |
| 21 | 21.668 | 4.09818 | 10.20% |
| 22 | 21.841 | 4.06605 | 4.80% |
| 23 | 22.589 | 3.93303 | 29.30% |
| 24 | 22.815 | 3.89464 | 23.60% |
| 25 | 23.018 | 3.86072 | 14.50% |
| 26 | 23.48 | 3.78575 | 23.60% |
| 27 | 23.757 | 3.74234 | 11.10% |
| 28 | 24.314 | 3.65785 | 44.50% |
| 29 | 25.687 | 3.46536 | 5.90% |
| 30 | 25.999 | 3.42447 | 8.70% |
| 31 | 26.978 | 3.30231 | 2.80% |
| 32 | 27.264 | 3.26836 | 5.30% |
| 33 | 29.69 | 3.00656 | 3.90% |
| 34 | 30.864 | 2.89488 | 2.00% |
| 35 | 31.61 | 2.82821 | 5.10% |
| 36 | 32.314 | 2.76819 | 2.10% |
| 37 | 34.559 | 2.59334 | 3.10% |
| 38 | 34.85 | 2.5723 | 4.30% |
| 39 | 37.55 | 2.39331 | 1.60% |

The mono-tosylate salt (Exp. 2-2) is also analyzed by DSC/TGA and HPLC, the results are summarized in table 4. DSC/TGA analysis shows an endothermic event at Tₒₙₛₑₜ=179.2° C., Tₚₑₐₖ=180.9° C. and ΔE=−77.1 J/g, and an exothermic event at Tₒₙₛₑₜ=277.1° C., Tₚₑₐₖ=285.3° C. and ΔE=+151.5 J/g. Analysis of the HPLC data shows a purity of 91-area %. Analysis of the ¹H-NMR data shows shift compared to the free base, both the free base and toluenesulfonic acid are present in a 1:1 molar ratio, which confirms the salt formation.

### Example 3: Bis-Tosylate Salt Crystal

Toluenesulfonic acid was added in a 1:2 molar ratio to a solution of d₂-lumateperone free base in 2-butanone. The solution was heated to 50° C., kept at this temperature for 1 hour, then cooled to room temperature. A greenish/brownish slurry was obtained which was isolated as an off-white solid. The experiment was performed at 1000 mg scale with a concentration of 50 mg/mL. The off-white solid is characterized by XRPD, DSC, TGA, FT-IR, LC and 1H-NMR, and the results are summarized in the table below:

| | | | | TGA | FT-IR | FT-IR | | |
|---|---|---|---|---|---|---|---|---|
| | | | DSC | mass | correlation | correlation | LC | |
| | | | Tₚₑₐₖ | loss | with FB | with CI | purity | |
| Sample # | Appearance | XRPD | (° C.) | (%) | (%) | (%) | (area-%) | ¹H-NMR |
| 3-1 | Off-white | Crystal | 186.6 | 1.0 | 6 | 18 | 94 | Ratio 1:2 |
| | solid | | | | | | | |

The XRPD pattern for the salt is shown in FIG. 3. The peaks are identified in tabular form in the table below:

TABLE 3
XRPD (Cu anode, Ni filter) for Bis-tosylate Salt Crystal

| # | Angle | d Value | Rel. Intensity |
|---|---|---|---|
| 1 | 4.146 | 21.29319 | 19.30% |
| 2 | 6.223 | 14.19209 | 1.10% |
| 3 | 8.306 | 10.63621 | 3.10% |
| 4 | 10.401 | 8.49836 | 14.30% |
| 5 | 12.209 | 7.24368 | 1.60% |
| 6 | 13.675 | 6.47004 | 7.90% |
| 7 | 14.16 | 6.24983 | 30.80% |
| 8 | 14.513 | 6.09848 | 25.00% |
| 9 | 14.905 | 5.93889 | 33.40% |
| 10 | 15.375 | 5.75839 | 43.40% |
| 11 | 15.901 | 5.56892 | 35.90% |
| 12 | 16.473 | 5.37695 | 24.20% |
| 13 | 17.093 | 5.1833 | 66.90% |
| 14 | 17.836 | 4.96894 | 13.50% |
| 15 | 17.941 | 4.94014 | 22.10% |
| 16 | 18.675 | 4.74767 | 23.10% |
| 17 | 19.193 | 4.62071 | 6.10% |
| 18 | 19.802 | 4.47986 | 6.10% |
| 19 | 20.399 | 4.35002 | 100.00% |
| 20 | 20.73 | 4.28143 | 58.50% |
| 21 | 21.301 | 4.16792 | 16.90% |
| 22 | 21.568 | 4.11697 | 11.70% |
| 23 | 22.437 | 3.95936 | 11.40% |
| 24 | 22.921 | 3.8769 | 11.60% |
| 25 | 23.198 | 3.83126 | 4.50% |
| 26 | 23.968 | 3.7098 | 22.40% |
| 27 | 24.586 | 3.61791 | 4.30% |
| 28 | 25.135 | 3.54015 | 12.60% |
| 29 | 25.895 | 3.43789 | 5.70% |
| 30 | 26.033 | 3.42 | 3.90% |
| 31 | 26.813 | 3.32231 | 6.40% |
| 32 | 27.336 | 3.25991 | 22.90% |
| 33 | 27.649 | 3.22376 | 15.80% |
| 34 | 28.582 | 3.12061 | 3.30% |
| 35 | 28.945 | 3.08226 | 4.40% |
| 36 | 29.285 | 3.04721 | 4.10% |
| 37 | 30.134 | 2.96327 | 5.80% |
| 38 | 31.612 | 2.82804 | 1.40% |
| 39 | 32.161 | 2.78099 | 2.40% |
| 40 | 33.495 | 2.67321 | 1.60% |
| 41 | 34.567 | 2.59273 | 1.30% |
| 42 | 35.452 | 2.52998 | 2.70% |
| 43 | 39.727 | 2.26707 | 2.30% |
| 44 | 42.643 | 2.11854 | 1.60% |

DSC/TGA analysis shows an endothermic event at Tₒₙₛₑₜ=181.6° C., Tₚₑₐₖ=186.6° C. and ΔE=−84.4 J/g; and an exothermic event at Tₒₙₛₑₜ=235.0° C., Tₚₑₐₖ=261.1° C. and ΔE=+331.5 J/g. Analysis of the HPLC data shows a purity of 94-area %. Analysis of the 1H-NMR data shows shift compared to the free base, both the free base and toluenesulfonic acid are present in a 1:2 molar ratio, which confirms the salt formation.

### Example 4: Free Base-Isonicotinamide Co-Crystal

d₂-lumateperone free base and isonicotinamide were mixed in methanol at a concentration of 100 mg/ml of d₂-lumateperone free base (Exp. 4-1). A clear solution was formed which was shaken overnight, then the solution was evaporated to dryness and a sticky oily solid was obtained. The experiment was repeated using at a concentration of 200 mg/mL, and a similar procedure, resulting in a sticky brown solid (Exp. 4-2). The solid was confirmed as the free base-isonicotinamide co-crystal based on XRPD, TGA, DSC, FT-IR, LC and 1H-NMR. A third experiment was conducted using a concentration of 200 mg/mL, but in a 5:2 v/v mixture of methanol and water. The results are summarized in the table below:

| | | | | TGA | FT-IR | FT-IR | | |
|---|---|---|---|---|---|---|---|---|
| | | | DSC | mass | correlation | correlation | LC | |
| | | | Tₚₑₐₖ | loss | with FB | with CI | purity | |
| Expt. # | Appearance | XRPD | (° C.) | (%) | (%) | (%) | (area-%) | ¹H-NMR |
| 4-1 | Sticky/oily | N/A | N/A | N/A | N/A | N/A | N/A | N/A |
| 4-2 | Sticky | Co- | 150.7 | N/A | 80 | 11 | 64 | Ratio 1:2 |
| | brown solid | Crystal | | | | | | |
| 4-3 | Sticky | Co- | 150.0 | 1.8 | 10 | 25 | 67 | N/A |
| | brown/ | Crystal | | | | | | |
| | yellowish | | | | | | | |
| | solid | | | | | | | |

The XRPD patterns for the salt (Exp. 4-2) is shown in FIG. 4. The peaks are identified in tabular form in the table below:

TABLE 4
XRPD (Cu anode, Ni filter) for Free
Base-Isonicotinamide Co-Crystal

| # | Angle | d Value | Rel. Intensity |
|---|---|---|---|
| 1 | 11.358 | 7.78412 | 0.80% |
| 2 | 14.476 | 6.11379 | 1.90% |
| 3 | 18.419 | 4.81292 | 1.90% |
| 4 | 20.66 | 4.29569 | 2.80% |
| 5 | 21.928 | 4.05007 | 1.60% |
| 6 | 22.099 | 4.01919 | 2.60% |
| 7 | 22.912 | 3.87828 | 100.00% |
| 8 | 24.214 | 3.67263 | 1.90% |
| 9 | 28.509 | 3.12837 | 1.30% |
| 10 | 28.526 | 3.12655 | 1.30% |
| 11 | 29.856 | 2.99023 | 0.70% |
| 12 | 32.096 | 2.78645 | 0.90% |
| 13 | 32.121 | 2.78437 | 0.90% |
| 14 | 34.737 | 2.58043 | 0.60% |
| 15 | 34.811 | 2.57513 | 0.60% |
| 16 | 36.547 | 2.45668 | 1.30% |
| 17 | 36.572 | 2.45506 | 1.30% |

DSC/TGA analysis (Exp. 4-2) shows an endothermic event at Tₒₙₛₑₜ=143.2° C., Tₚₑₐₖ=150.7° C. and ΔE=−55.3 J/g. Analysis of the HPLC data shows a purity of 64-area %. Analysis of the ¹H-NMR data shows free base and isonicotinamide are present in a 1:2 molar ratio, which confirms the co-crystal formation.

### Example 5: Tosylate-Lysine Co-Crystal

d₂-lumateperone mono-tosylate (Exp. 2-3) and L-lysine free base were mixed in methanol. The solution turned into a gel overnight and additional methanol was added in order to create a slurry. The product was isolated as a yellowish powder and characterized by XRPD, DSC, TGA, FT-IR, LC and 1H-NMR. The results are summarized in the table below:

| | | | | TGA | FT-IR | FT-IR | | |
|---|---|---|---|---|---|---|---|---|
| | | | DSC | mass | correlation | correlation | LC | |
| | | | Tₚₑₐₖ | loss | with FB | with CF | purity | |
| Sample # | Appearance | XRPD | (° C.) | (%) | (%) | (%) | (area-%) | ¹H-NMR |
| 5-1 | Yellowish | Co- | 203.1 | 20.6 | N/A | N/A | 93.0 | Free base, |
| | powder | Crystal | 221.7 | | | | | tosylate |
| | | | | | | | | and lysine |

The XRPD patterns for the co-crystal is shown in FIG. 5. The peaks are identified in tabular form in the table below:

TABLE 5
XRPD (Cu anode, Ni filter) for Tosylate-Lysine Co-Crystal

| # | Angle | d Value | Rel. Intensity |
|---|---|---|---|
| 1 | 7.471 | 11.82319 | 5.90% |
| 2 | 9.541 | 9.26261 | 44.60% |
| 3 | 11.149 | 7.92974 | 3.30% |
| 4 | 11.522 | 7.67421 | 11.80% |
| 5 | 12.469 | 7.09293 | 8.10% |
| 6 | 14.969 | 5.9137 | 10.30% |
| 7 | 15.437 | 5.73524 | 12.70% |
| 8 | 17.543 | 5.05147 | 3.20% |
| 9 | 18.184 | 4.87469 | 22.40% |
| 10 | 19.14 | 4.63327 | 10.60% |
| 11 | 20 | 4.43594 | 100.00% |
| 12 | 20.177 | 4.39754 | 18.60% |
| 13 | 20.781 | 4.27107 | 13.90% |
| 14 | 22.386 | 3.96821 | 64.10% |
| 15 | 22.708 | 3.91277 | 21.00% |
| 16 | 23.152 | 3.83863 | 7.30% |
| 17 | 24.535 | 3.6254 | 8.30% |
| 18 | 24.857 | 3.57907 | 12.80% |
| 19 | 25.405 | 3.50311 | 2.10% |
| 20 | 25.913 | 3.43555 | 82.20% |
| 21 | 28.025 | 3.18128 | 1.40% |
| 22 | 28.91 | 3.08591 | 1.70% |
| 23 | 29.73 | 3.00261 | 7.90% |
| 24 | 30.211 | 2.95595 | 22.20% |
| 25 | 30.655 | 2.91408 | 4.30% |
| 26 | 30.901 | 2.8915 | 6.60% |
| 27 | 32.121 | 2.78434 | 4.10% |
| 28 | 33.567 | 2.66765 | 5.10% |
| 29 | 33.892 | 2.64284 | 4.70% |
| 30 | 34.195 | 2.62011 | 3.30% |
| 31 | 35.017 | 2.56045 | 2.90% |
| 32 | 35.857 | 2.50239 | 4.20% |
| 33 | 37.319 | 2.4076 | 4.70% |
| 34 | 39.287 | 2.29144 | 2.40% |
| 35 | 39.712 | 2.26788 | 1.20% |
| 36 | 40.68 | 2.2161 | 1.60% |
| 37 | 42.856 | 2.10851 | 1.00% |
| 38 | 43.764 | 2.06684 | 1.50% |

DSC/TGA analysis shows a first endothermic event at Tₒₙₛₑₜ=193.1° C., Tₚₑₐₖ=203.1° C. and ΔE=−72.2 J/g, and a second endothermic event at Tₒₙₛₑₜ=188.8° C., Tₚₑₐₖ=221.7° C. and ΔE=−109.3 J/g. Analysis of the HPLC data shows a purity of 93-area %. Analysis of the ¹H-NMR data shows d₂-lumateperone free base, toluenesulfonic acid, and lysine, which confirms the co-crystal formation.

1. 2,2-d₂-1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one (d₂-lumateperone), in the form of a salt selected from a hydrochloride, mono-tosylate, or bis-tosylate salt, or in the form of a co-crystal between d₂-lumateperone free base and isonicotinamide or between d₂-lumateperone tosylate and lysine free base (e.g., L-lysine), wherein said salt or co-crystal is in solid crystalline form.

2. The salt or co-crystal according to claim 1, wherein the salt or co-crystal is substantially free of other forms of d₂-lumateperone.

3. The salt or co-crystal according to claim 1 which is a hydrochloride salt crystal, e.g., having an X-ray diffraction pattern substantially corresponding to FIG. 1(a) or 1(b).

4. The salt or co-crystal according to claim 1 which is a mono-tosylate salt crystal, e.g., having an X-ray diffraction pattern substantially corresponding to FIG. 2.

5. The salt or co-crystal according to claim 1 which is a bis-tosylate salt crystal, e.g., having an X-ray diffraction pattern substantially corresponding to FIG. 3.

6. The salt or co-crystal according to claim 1 which is a co-crystal between d₂-lumateperone free base and isonicotinamide, e.g., having an X-ray diffraction pattern substantially corresponding to FIG. 4.

7. The salt or co-crystal according to claim 1 which is a co-crystal between d₂-lumateperone mono-tosylate and lysine free base (e.g., L-lysine), e.g., having an X-ray diffraction pattern substantially corresponding to FIG. 5.

8. A salt according to claim 1, wherein the salt comprises a 1:1 molar ratio of d₂-lumateperone and hydrochloric acid.

9. A co-crystal according to claim 1, wherein the co-crystal comprises a 1:1 or 2:1 molar ratio of d₂-lumateperone free base and isonicotinamide.

10. A method making a salt according to claim 1, comprising
(a) reacting free base d₂-lumateperone with an acid selected from hydrochloric acid and toluenesulfonic acid, e.g., together with an organic solvent, and
(b) recovering the salt thus formed.

11. A method making a co-crystal according to claim 1, comprising
(a) combining free base d₂-lumateperone with isonicotinamide, or d₂-lumateperone mono-tosylate with lysine free base (e.g., L-lysine), e.g., together with an organic solvent, and
(b) recovering the salt thus formed.

12. A method of purifying 2,2-d₂-1-(4-fluorophenyl)-4-((6bR,10aS)-3-methyl-2,3,6b,7,10,10a-hexahydro-1H-pyrido[3′,4′:4,5]pyrrolo[1,2,3-de]quinoxalin-8(9H)-yl)butan-1-one (d₂-lumateperone) in free or salt form, comprising reacting d₂-lumateperone with an acid selected from hydrochloric acid and toluenesulfonic acid, or combining free base d₂-lumateperone with isonicotinamide, or d₂-lumateperone mono-tosylate with lysine free base (e.g., L-lysine), recovering the salt or co-crystal thus formed, and optionally converting the salt or co-crystal back to d₂-lumateperone free base or to another salt form.

13. A method for the prophylaxis or treatment of a human suffering from a disease or abnormal condition involving or mediated by the 5-HT_{2A} receptor, serotonin transporter (SERT), and/or dopamine D₁/D₂ receptor signaling pathways comprising administering to said human an effective amount of a salt according to claim 1.

14. A pharmaceutical composition comprising a salt according to claim 1, in combination or association with a pharmaceutically acceptable diluent or carrier.

15. A salt according to claim 1, wherein the salt is a mono-tosylate salt.

16. A salt according to claim 1, wherein the salt is a bis-tosylate salt.

17. A co-crystal according to claim 1, wherein the co-crystal comprises a 1:1 molar ratio of d₂-lumateperone mono-tosylate and L-lysine free base.
